(19) United States
(12) Patent Application Publication
(10) Pub. No.: US 2015/0057575 A1
(43) Pub. Date: Feb. 26, 2015

(54) FORCE MEASUREMENT APPARATUS AND FORCE MEASUREMENT METHOD, MASTER SLAVE APPARATUS, FORCE MEASUREMENT PROGRAM, AND INTEGRATED ELECTRONIC CIRCUIT

(71) Applicant: Panasonic Intellectual Property Management Co., Ltd., Osaka (JP)

(72) Inventors: Yuko TSUSAKA, Osaka (JP); Taichi SATO, Kyoto (JP); Yudai FUDABA, Osaka (JP)

(21) Appl. No.: 14/528,440

(22) Filed: Oct. 30, 2014

Related U.S. Application Data
(63) Continuation of application No. PCT/JP2013/003843, filed on Jun. 20, 2013.

(30) Foreign Application Priority Data
Jul. 10, 2012 (JP) 2012-154548

(51) Int. Cl. A61B 19/00; A61M 25/09; A61B 1/00

(52) U.S. Cl. CPC A61B 19/46; A61B 1/00009; A61M 25/09; A61B 1/00055; A61M 2025/09183; A61B 2019/465
USPC 600/587

(57) ABSTRACT

A force measurement apparatus that, when an insertion member is inserted into a living body vessel, measures a force at time the insertion member contacts with living body vessel, includes a force detector that measures, from an outside of a body, a force applied from the insertion member to the living body vessel, a reference point calculating unit that calculates a time point when a force applied from the insertion member into the living body vessel is individually measured based on information about the force detected by the force detector during the insertion of the insertion member into the living body vessel, and an individual force calculating unit that individually calculates the force applied from the insertion member to the living body vessel based on information about the time point and information about the force detected by the force detector.

### CROSS-REFERENCE TO RELATED APPLICATIONS

This is a continuation application of International Application No. PCT/JP2013/003843, with an international filing date of Jun. 20, 2013, which claims priority of Japanese Patent Application No.: 2012-154548 filed on Jul. 10, 2012, the content of which is incorporated herein by reference.

### TECHNICAL FIELD

The technical field relates to a force measurement apparatus and a force measurement method, a master slave apparatus, a force measurement program, and an integrated electronic circuit, each of which is used for assisting an operator's procedure when an insertion member that is a catheter or an endoscope is inserted into a living body vessel.

### BACKGROUND ART

In recent years, while a fluoroscopic image or the like is being viewed, a linear insertion member such as a guide wire or a catheter is inserted into a living body vessel of a human body such as a blood vessel so that an operative method such as a medical treatment on an angiostenosis part is performed. An operator checks a state of a living body vessel or an insertion member through a photographed image and at the same time directly feels force sensitive information about insertion resistance caused by contact between the insertion member and the living body vessel by the operator oneself. When an insertion member is manipulated outside a body, the insertion member might occasionally damage a duct. Further, only the operator can check the force sensitive information about the insertion resistance caused by the contact between the insertion member and the living body vessel of a human body, but the force sensitive information cannot be quantitatively checked as numerical values.

In order to solve such a problem, a method for measuring deflection of the insertion member so as to measure insertion resistance to be applied to the insertion member from the outside of a human body is present (see Patent Literature 1). This system enables the insertion resistance, which has been checked by operator's intuition, to be quantitatively checked by measuring the insertion resistance applied to the insertion member.

### CITATION LIST

### Patent Literature

[Patent Literature 1] Japanese Unexamined Patent Publication No. 2009-139179

### SUMMARY OF THE INVENTION

However, Patent Literature 1 describes the method in which a sensor is not provided directly to the insertion member. With this method, a force caused by contact of a distal end of an insertion member from the outside of the body or a frictional force caused by contact between the middle of the insertion member and a living body vessel can be totally measured as force sensitive information. However, when a lot of meandering portions are present, the frictional force increases, and thus a load on the living body vessel cannot be detected by using a predetermined threshold value. Further, since the force information measured outside the human body is the force sensitive information obtained by summing up the force caused by contact of the distal end of an insertion member or the frictional force caused by contact between the middle of the insertion member and the living body vessel, a force to be applied to the distal end of the insertion member or the force to be applied at time of passing through each of the meandering portions cannot be individually measured.

One non-limiting and exemplary embodiment provides a force measurement apparatus and a force measurement method, a master slave apparatus, a force measurement program, and an integrated electronic circuit, each of which can individually estimate a force to be applied to a distal end of an insertion member or a force to be applied to each of meandering portions based on force information measured outside a human body.

Additional benefits and advantages of the disclosed embodiments will be apparent from the specification and Figures. The benefits and/or advantages may be individually provided by the various embodiments and features of the specification and drawings disclosure, and need not all be provided in order to obtain one or more of the same.

In one general aspect, the techniques disclosed here feature: A force measurement apparatus that, when an insertion member that is a catheter or an endoscope is inserted into a living body vessel, measures a force at time when the insertion member contacts with the living body vessel, the apparatus comprising:

a force detector that measures, from an outside of the living body vessel, a force generated during the insertion of the insertion member into the living body vessel;

an individual force calculation parameter determining unit that determines a time point when the force generated during the insertion of the insertion member into the living body vessel is individually measured or an insertion length at that time point as an individual force calculation parameter based on information about the force detected by the force detector during the insertion of the insertion member into the living body vessel; and

an individual force calculating unit that individually calculates the force generated during the insertion of the insertion member into living body vessel at each time point or each insertion length as an individual force based on the time point or information about the insertion length at that time point and the information about the force detected by the force detector as the individual force calculation parameter determined by the individual force calculation parameter determining unit.

These general and specific aspects may be implemented using a system, a method, and a computer program, and any combination of systems, methods, and computer programs.

With the force measurement apparatus and the force measurement method, the master slave apparatus, the force measurement program, and the integrated electronic circuit from the aspect of the present invention, forces that are generated when the insertion member is inserted into a duct are not measured as a sum but can be measured at individual contact portions. Further, the use of the force measurement apparatus enables manipulation assist for stopping a robot when a load is applied.

### BRIEF DESCRIPTION OF THE DRAWINGS

These and other aspects and features of the present disclosure will become clear from the following description taken in conjunction with the embodiments thereof with reference to the accompanying drawings, in which:

FIG. 1 is a view illustrating a schematic constitution of a force measurement apparatus according to a first embodiment of the present invention;

FIG. 2 is a block diagram illustrating a detailed constitution of the force measurement apparatus according to the first embodiment of the present invention;

FIG. 3 is a view relating to measurement information database according to the first embodiment of the present invention;

FIG. 4A is a view illustrating the schematic constitution of the force measurement apparatus according to the first embodiment of the present invention;

FIG. 4B is a view illustrating the schematic constitution of the force measurement apparatus according to the first embodiment of the present invention;

FIG. 4C is a view illustrating the schematic constitution of the force measurement apparatus according to the first embodiment of the present invention;

FIG. 4D is a view illustrating a correspondence table of a deflection quantity and a force in the force measurement apparatus according to the first embodiment of the present invention;

FIG. 4E is a view illustrating a schematic constitution of an insertion length detector according to the first embodiment of the present invention;

FIG. 4F is a view illustrating a correspondence table of a number of marks and an insertion quantity of the insertion length detector according to the first embodiment of the present invention;

FIG. 5 is a view describing one example of a decided result notification unit according to the first embodiment of the present invention;

FIG. 6 is a flowchart of the force measurement apparatus according to the first embodiment of the present invention;

FIG. 7 is an explanatory view describing a catheter inserting motion according to the first embodiment of the present invention, (A) of FIG. 7 is a graph illustrating a relationship between a force and an insertion length at catheter inserting time, and (B) to (E) of FIG. 7 are views describing the catheter inserting motion;

FIG. 8 is a graph illustrating a relationship between the force and the insertion length at the catheter inserting time according to the first embodiment of the present invention;

FIG. 9 is a view relating to threshold value data of the force measurement apparatus according to the first embodiment of the present invention;

FIG. 10 is an explanatory view describing the catheter insertion work according to the first embodiment of the present invention;

FIG. 11 is a block diagram illustrating a detailed constitution of the force measurement apparatus according to a second embodiment of the present invention;

FIG. 12 is an explanatory view describing the catheter inserting motion according to the second embodiment of the present invention, (A) of FIG. 12 is a graph illustrating a relationship between the force and the insertion length at the catheter inserting time, and (B) to (G) of FIG. 12 are views describing the catheter inserting motion;

FIG. 13 is a flowchart of the force measurement apparatus according to the second embodiment of the present invention;

FIG. 14 is a graph illustrating the relationship between the force and the insertion length at the catheter inserting time according to the second embodiment of the present invention;

FIG. 15 is a view relating to measurement information database according to the second embodiment of the present invention;

FIG. 16 is an explanatory view describing the catheter inserting motion according to the second embodiment of the present invention, (A) of FIG. 16 is a graph illustrating the relationship between the force and the insertion length at the catheter inserting time, and (B) to (G) of FIG. 16 are views describing the catheter inserting motion;

FIG. 17 is a graph illustrating the relationship between the force and the insertion length at the catheter inserting time according to the second embodiment of the present invention;

FIG. 18A is a view relating to measurement information database according to the second embodiment of the present invention;

FIG. 18B is a view relating to the measurement information database according to the second embodiment of the present invention;

FIG. 19 is a view illustrating a schematic constitution of a master slave apparatus according to a third embodiment of the present invention;

FIG. 20 is a block diagram illustrating a detailed constitution of the master slave apparatus according to the third embodiment of the present invention;

FIG. 21 is a flowchart of the master slave apparatus according to the third embodiment of the present invention;

FIG. 22 is a view describing the catheter insertion work according to the third embodiment of the present invention;

FIG. 23 is a block diagram illustrating a detailed constitution of the master slave apparatus according to the fourth embodiment of the present invention;

FIG. 24 is a flowchart of the master slave apparatus according to a fourth embodiment of the present invention;

FIG. 25 is a view describing a slave motion generating unit according to the fourth embodiment of the present invention;

FIG. 26 is a view illustrating a schematic constitution of the force measurement apparatus according to a fifth embodiment of the present invention;

FIG. 27 is a block diagram illustrating a detailed constitution of the force measurement apparatus according to the fifth embodiment of the present invention;

FIG. 28 is a view describing one example of the decided result notification unit according to the fifth embodiment of the present invention;

FIG. 29 is a view describing information about force decided results according to the fifth embodiment of the present invention;

FIG. 30 is a view describing notification information according to the fifth embodiment of the present invention;

FIG. 31 is a view relating to a control information database according to the fifth embodiment of the present invention; and

FIG. 32 is a flowchart of the force measurement apparatus according to the fifth embodiment of the present invention.

### DETAILED DESCRIPTION

Examples of the disclosed technique are as follows.

1st aspect: A force measurement apparatus that, when an insertion member that is a catheter or an endoscope is inserted into a living body vessel, measures a force at time when the insertion member contacts with the living body vessel, the apparatus comprising:

a force detector that measures, from an outside of the living body vessel, a force generated during the insertion of the insertion member into the living body vessel;

an individual force calculation parameter determining unit that determines a time point when the force generated during the insertion of the insertion member into the living body vessel is individually measured or an insertion length at that time point as an individual force calculation parameter based on information about the force detected by the force detector during the insertion of the insertion member into the living body vessel; and

an individual force calculating unit that individually calculates the force generated during the insertion of the insertion member into living body vessel at each time point or each insertion length as an individual force based on the time point or information about the insertion length at that time point and the information about the force detected by the force detector as the individual force calculation parameter determined by the individual force calculation parameter determining unit.

This constitution enables a force to be applied to each of contact portions to be estimated based on the force measured outside the living body vessel.

2nd aspect: The force measurement apparatus according to the 1st aspect, wherein when the insertion member is inserted into the living body vessel, the individual force calculation parameter determining unit determines a time point or an insertion length where displacement of the force is a predetermined threshold value or more, as the individual force calculation parameter at each predetermined insertion length,

the individual force calculating unit adds a value, which is obtained by dividing a value, which is obtained by subtracting information about the force at the immediately preceding time point or at an insertion length at that time point from the information about the force detected by the force detector at a measurement time point or at an insertion length at that time point, by a number of the time points or the insertion lengths determined until the measurement time point or the insertion length, to the individual force at each time point or each insertion length.

This constitution enables a force to be applied to each of contact portions to be estimated based on the force measured outside the living body vessel.

3rd aspect: The force measurement apparatus according to the 1st or 2nd aspect, further comprising: a correcting unit that, when after the insertion member is inserted into the living body vessel and is once partially pulled back, the insertion member is again inserted into the living body vessel, makes a correction so that the time points or the insertion lengths, which are already determined by the individual force calculation parameter determining unit between a pulling-back start time point or an insertion length at that time point and a reinsertion time point or an insertion length at that time point, are deleted, wherein

the individual force calculating unit calculates individual forces based on the time point or the insertion length corrected by the correcting unit.

This constitution enables a force to be applied to each of contact portions to be estimated based on the force measured outside the living body vessel.

4th aspect: The force measurement apparatus according to any one of the 1st to 3rd aspects, further comprising a force deciding unit that, when information about forces of the predetermined threshold value or more in the information about the individual forces calculated by the individual force calculating unit is present, decides that a load is generated in the living body vessel or the insertion member.

With this constitution, a decision can be made whether a load is applied to the living body vessel or the insertion member.

5th aspect: The force measurement apparatus according to any one of the 1st to 4th aspects, further comprising: an imaging device that images an image of a portion of the living body vessel into which the insertion member is inserted; and

a decided result notification unit that adds the individual forces calculated by the individual force calculating unit or a decided result decided by the force deciding unit to the image obtained by imaging the living body vessel or the insertion member so as to display the image.

This constitution enables whether a load is applied to the living body vessel or the insertion member to be displayed together with an image.

6th aspect: The force measurement apparatus according to any one of the 1st to 5th aspects, further comprising: an output unit that notifies an operator of the individual forces calculated by the individual force calculating unit or the decided result decided by the force deciding unit as a sound or an image.

This constitution enables whether a load is applied to the living body vessel or the insertion member to be checked through a voice or the like.

7th aspect: The force measurement apparatus according to any one of the 1st to 4th aspects, further comprising:

a notification information determining unit that determines information to be notified based on the decided result decided by the force deciding unit;

an imaging device that images an image of the portion of the living body vessel into which the insertion member is inserted based on the notification information determined by the notification information determining unit;

an imaging device controller that controls the imaging device; and

a decided result notification unit that adds the notification information determined by the notification information determining unit to the image imaged by the imaging device under control of the imaging device controller so as to display the image.

When a load is applied to the living body vessel or the insertion member, this constitution enables the load to be checked together with an image imaged by an imaging device.

8th aspect: A master slave apparatus comprising a slave mechanism that delivers an insertion member that is a catheter or an endoscope to a living body vessel, and a master mechanism with which a person remotely manipulates the slave mechanism, said apparatus comprising:

a force measurement apparatus comprising:

a force detector that measures, from an outside of the living body vessel, a force generated during insertion of the insertion member into the living body vessel;

an individual force calculation parameter determining unit that determines a time point when the force generated during the insertion of the insertion member into the living body vessel is individually measured or an insertion length at that time point as an individual force calculation parameter based on information about the force detected by the force detector during the insertion of the insertion member into the living body vessel; and

an individual force calculating unit that individually calculates the force generated during the insertion of the insertion member into the living body vessel at each time point or at each insertion length as an individual force based on information about the time point or the insertion length at that time point that is the individual force calculation parameter determined by the individual force calculation parameter determining unit and the information about the force detected by the force detector;

a force transmission portion determining unit that determines a force to be transmitted to the master mechanism based on force information obtained by the force measurement apparatus;

a force correcting unit that, when switching into the force determined by the force transmission portion determining unit, makes a correction in a manner that smoothing is conducted on the force so that the force is smoothly switched;

a master controller with which the person manipulates the master mechanism based on the force information of the force measurement apparatus so as to convert manipulation information about the master mechanism into an electric signal; and

a slave controller that is connected to the slave mechanism and the master controller and outputs a control signal that transmits the manipulation information about the master mechanism sent from the master controller to the slave mechanism, and transmits the force information corrected by the force correcting unit to the master controller.

This constitution enables a force(s) only on a necessary portion(s) to be transmitted to the master mechanism.

9th aspect: The master slave apparatus according to the 8th aspect, wherein the force measurement apparatus further comprises:

a force deciding unit that, when the information about forces that is the predetermined threshold value or more is present in the information about the individual forces calculated by the individual force calculating unit, decides that a load is generated in the living body vessel or the insertion member; and

a slave motion generating unit that generates a motion for stopping the slave motion when the force deciding unit decides that the force information is the predetermined threshold value or more,

the slave controller controls the slave mechanism based on the motion generated by the slave motion generating unit.

When a load is applied to the living body vessel or the insertion member, this constitution enables the slave mechanism to be controlled to be stopped.

10th aspect: The master slave apparatus according to the 8th or 9th aspect, further comprising: a slave motion generating unit that sets one of or both of a vibration cycle and an amplitude of vibration for vibrating the slave according to a level of the information about the force measured by the measurement apparatus so as to generate motion of the slave, wherein

the slave controller controls the slave mechanism based on the motion generated by the slave motion generating unit.

When a load is applied to the living body vessel or the insertion member and thus the insertion member cannot advance, this constitution enables the advancing through suitable vibration control.

11th aspect: A force measurement method for, when an insertion member that is a catheter or an endoscope is inserted into a living body vessel, measuring a force at time the insertion member contacts with the living body vessel, the method comprising;

measuring, from an outside of the living body vessel, a force generated during insertion of the insertion member into the living body vessel, using a force detector;

determining a time point when the force generated during the insertion of the insertion member into the living body vessel is individually measured or an insertion length at that time point as an individual force calculation parameter based on information about the force detected by the force detector during the insertion of the insertion member into the living body vessel, using an individual force calculation parameter determining unit; and

individually calculating the force generated during the insertion of the insertion member into the living body vessel at each time point or at each insertion length as an individual force based on information about the time point or the insertion length at that time point determined as the individual force calculation parameter by the individual force calculation parameter determining unit and the information about the force detected by the force detector, using an individual force calculating unit.

This constitution enables a force to be applied to each of contact portions to be estimated based on the force measured outside the living body vessel.

12th aspect: A force measurement program that, when an insertion member that is a catheter or an endoscope is inserted into a living body vessel, measures a force at time the insertion member contacts with the living body vessel,

the program allowing a computer to function as:

an individual force calculation parameter determining unit that determines a time point a force generated during insertion of the insertion member into the living body vessel is individually measured or an insertion length at that time point as an individual force calculation parameter during the insertion of the insertion member into the living body vessel based on information about a force detected by a force detector that measures, from an outside of the living body vessel, the force generated during the insertion of the insertion member into the living body vessel; and

an individual force calculating unit that individually calculates the force generated during the insertion of the insertion member into living body vessel at each time point or each insertion length as an individual force based on information about the time point or the insertion length at that time point that is determined as the individual force calculation parameter by the individual force calculation parameter determining unit and the information about the force detected by the force detector.

This constitution enables a force to be applied to each of contact portions to be estimated based on the force measured outside the living body vessel.

13th aspect: An integrated electronic circuit that, when an insertion member that is a catheter or an endoscope is inserted into a living body vessel, measures a force at time the insertion member contacts with the living body vessel, the integrated electronic circuit comprising:

an individual force calculation parameter determining unit that determines a time point when a force generated during the insertion of the insertion member into the living body vessel is individually measured or an insertion length at that time point as an individual force calculation parameter during the insertion of the insertion member into the living body vessel based on information about a force detected by a force detector that measures, on an outside of the living body vessel, the force generated during the insertion of the insertion member into the living body vessel during the insertion of the insertion member into the living body vessel; and

an individual force calculating unit that individually calculates the force generated during the insertion of the insertion member into living body vessel at each time point or each insertion length as an individual force based on information about the time point or the insertion length at that time point determined as the individual force calculation parameter by the individual force calculation parameter determining unit and the information about the force detected by the force detector.

This constitution enables a force to be applied to each of contact portions to be estimated based on the force measured outside the living body vessel.

### First Embodiment

A summary of a force measurement apparatus 1 according to the first embodiment of the present invention is described first.

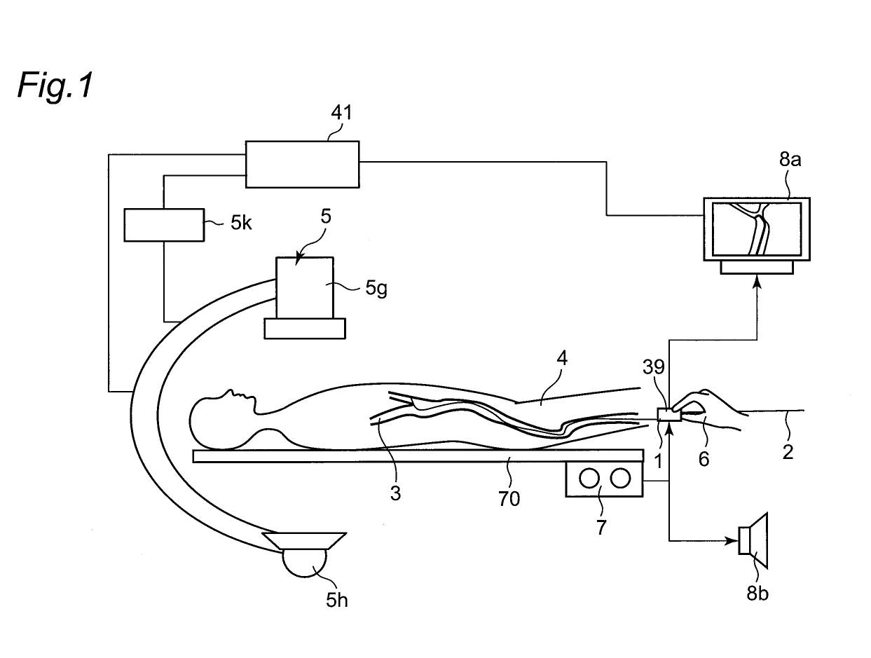

FIG. 1 illustrates a state of catheterization study or treatment with which an operator 6 inserts a guide wire 2 as one example of an insertion member into an affected area of a blood vessel 3 of a brain or a heart as one example of a living body vessel of a human body 4, from the outside of the human body.

A portion opposite to the distal end of the guide wire 2 is gripped and fixed to a torque device 39, and the operator 6 grips the torque device 39 so as to insert the guide wire 2. While the operator 6 is inserting the guide wire 2 into the blood vessel 3, an X-ray imaging device 5 as one example of an imaging device images the blood vessel 3 or the guide wire 2 from the outside of the human body, and a monitor 8a displays an image imaged by the X-ray imaging device 5. The X-ray imaging device 5 has an X-ray generator 5g, and an X-ray detector 5h related to the X-ray generator 5g. The X-ray generator 5g emits radioactive rays (for example, X rays) to a portion of the human body 4 on a bed 70 to be imaged, and the X-ray detector 5h detects an X-ray image transmitted through the human body 4. The X-ray image detected by the X-ray detector 5h is connected to the monitor 8a via an X-ray imaging controller 41 as one example of an imaging device controller, so as to be displayed on the monitor 8a. The X-ray imaging controller 41 controls to drive an X-ray imaging device transfer unit 5k so as to be capable of transferring the X-ray generator 5g and the X-ray detector 5h to a portion that needs to be imaged as need arises. The following embodiments can employ the similar constitution.

The force measurement apparatus 1 is arranged on the distal end of a torque device 39, and individually measures a force that are generated when the operator 6 inserts the guide wire 2, such as a contact force that is generated when the guide wire 2 contacts with the blood vessel 3 or a frictional force that is generated when the guide wire 2 contacts with a meandering portion or a branch portion of the blood vessel 3. When a load is applied to the blood vessel 3, the force measurement apparatus 1 notifies the operator of warning through the monitor 8a or a speaker 8b as one example of an output unit.

The operator inserts the guide wire 2 while checking the X-ray image displayed on the monitor 8a or the warning from the speaker 8b. Further, an input IF (interface) 7 is an operating interface for instructing start and end of the detection in the force measurement apparatus 1, and is composed of, for example, buttons. Upon receiving the instructions for starting the force measurement from the input IF 7, a force measurement controller 200 starts a force measuring process in the force measurement apparatus 1. In the meanwhile, upon receiving the instructions for ending the force measurement from the input IF 7, the force measurement controller 200 ends the force measuring process in the force measurement apparatus 1. The force measurement controller 200 controls also start and end of the imaging operation in the X-ray imaging device 5 via the X-ray imaging controller 41 based on the instructions for starting and ending the force measurement.

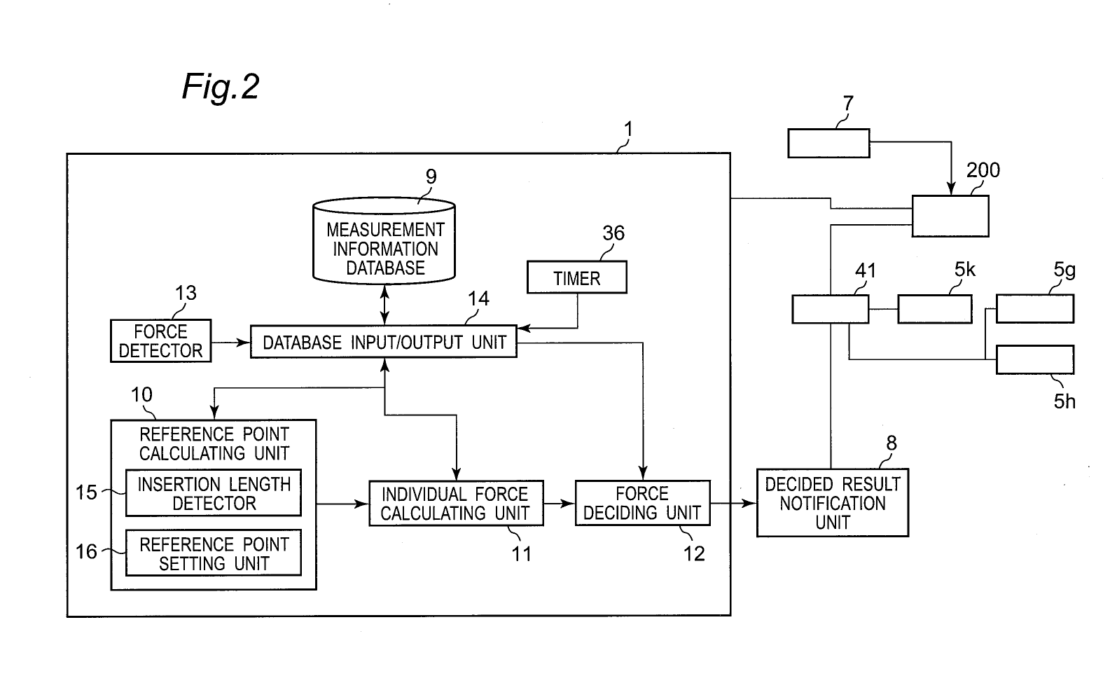

FIG. 2 illustrates a constitution of the force measurement apparatus 1.

The force measurement apparatus 1 according to the first embodiment is composed of at least a force detector 13, a reference point calculating unit 10 that functions as one example of a parameter determining unit for individual force calculation or a time point calculating unit, and an individual force calculating unit 11. Besides the above devices, the force measurement apparatus 1 according to the first embodiment includes a database input/output unit 14, a measurement information database 9, a force deciding unit 12, a decided result notification unit 8, and a timer 36.

<<Force Detector 13>>

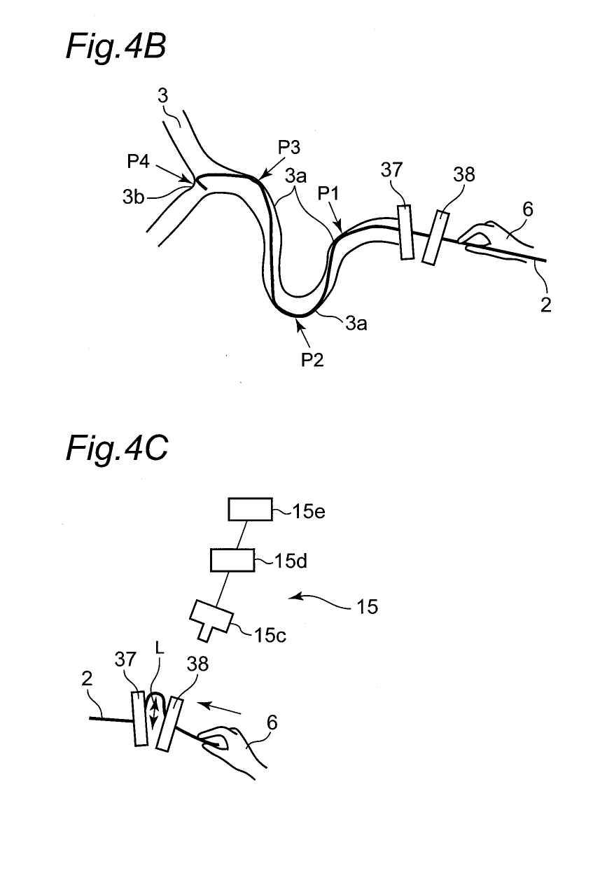

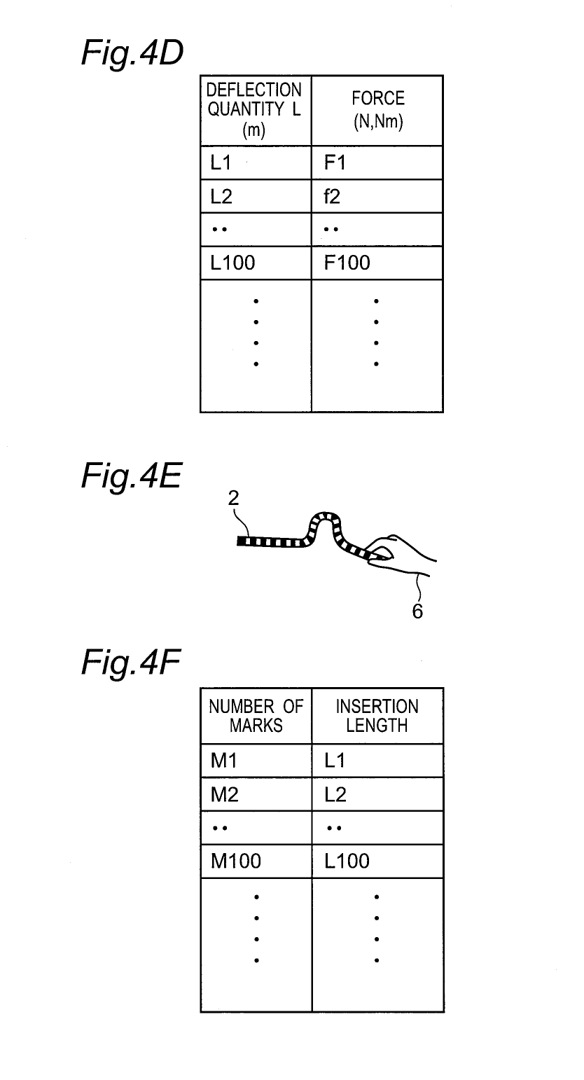

The force detector 13 detects a force, which acts (is generated) on the guide wire 2 when the guide wire 2 comes in contact with the blood vessel 3 from the outside of the human body 4, while the guide wire 2 as one example of the insertion member is being inserted into the blood vessel 3 as one example of the living body vessel (the time including not only the time when the start of the insertion of the guide wire 2 into the blood vessel 3 but also the time during the insertion), on the outside of the blood vessel 3. For example, the force detector 13 is composed of a six-axis force sensor for measuring a force in an insertion direction of the guide wire 2. As shown in FIG. 4A, the force detector 13 is arranged on a distal end of the torque device 39. The operator 6 grips the torque device 39 to manipulate the guide wire 2, and when the guide wire 2 contacts with each of meandering portions 3a or branch portions 3b of the blood vessel 3, the force detector 13 sums up the force at each meandering portions 3a or each branch point 3b to measure the force.

For example, when forces P1, P2, P3, and P4 are generated at the meandering portions 3a or the branch points 3b, respectively, as shown in FIG. 4A, the force detector 13 cannot individually detect the forces P1, P2, P3, and P4, and calculates a summed up value of the forces P1, P2, P3, and P4 (in this example, P1+P2+P3+P4 Pt) Pt. The value Pt detected by the force detector 13 is detected by the force detector 13 by using the timer 36, described later, at every constant time (for example, every 4 msec), and the detected force Pt is output together with a time to the database input/output unit 14, described later, from the force detector 13, so as to be stored from the database input/output unit 14 into the measurement information database 9.

The force detector 13 according to the first embodiment is the six-axis force sensor, but the force detector 13 may be a force sensor that enables measurement on two axes in the insertion direction of the guide wire 2 and a rotating direction around the insertion direction. Further, the force detector 13 is arranged at the distal end of the torque device 39, but for example as shown in FIG. 4B, the guide wire 2 is allowed to pass through a first fixing portion 37 and a second fixing portion 38, and when the operator applies a force as shown in FIG. 4C, a deflection quantity (a length L in FIG. 4C) of the two first and second fixing portions 37 and 38 is measured by an image recognition apparatus 15c such as a laser displacement meter or a camera. A table (shown in FIG. 4D) showing a relationship between the deflection quantity L and the force that is prepared in advance is used in a second calculating unit 15e for the insertion length detector, and the force related to the deflection quantity may be calculated by the second calculating unit 15e for the insertion length detector.

<<Timer 36>>

The timer 36 makes the database input/output unit 14 perform an operation after certain constant time passes (for example, every 4 msec).

<<Database Input/Output Unit 14>>

The database input/output unit 14 inputs/outputs data with the measurement information database 9, the force detector 13, the reference point calculating unit 10, the individual force calculating unit 11, and the force deciding unit 12.

<<Reference Point Calculating Unit 10>>

The reference point calculating unit 10 has an insertion length detector 15, and a reference point setting unit 16 that functions as one example of a time point setting unit, and determines a time point or an insertion length at that time point as an individual force calculation parameter. As a representative example, an example in which the individual force calculating unit 11 determines a time point as the individual force calculation parameter and calculates an individual force using the determined individual force calculation parameter as described later is described below. As a modification example, instead of the time point, an insertion length at that time point is determined as the individual force calculation parameter, and the individual force calculating unit 11 may calculate an individual force using the determined individual force calculation parameter.

The insertion length detector 15 is arranged on the torque device 39 to be manipulated by the operator 6 outside the body as shown in FIG. 4A, for example. As a concrete constitution, the insertion length detector 15 is composed of a distance sensor 15a and a first calculating unit 15b for the insertion length detector. The position of the torque device 39 is measured by the distance sensor 15a, a transfer distance of the torque device 39 from a position before transfer is obtained by the first calculating unit 15b for the insertion length detector based on information about the measured result. In such a manner, the transfer distance is detected as the insertion length by the first calculating unit 15b for the insertion length detector.

In the first embodiment, the insertion length detector 15 is arranged on the torque device 39, but the detector 15 is not limited to this. For example, as another example of the insertion length detector 15, contrasting (for example, black and white) marks are given to the guide wire 2 as shown in FIG. 4E, and a number of marks is imaged by a camera 15c, so that an imaged image is recognized by an image recognizing unit 15d. As a result, the marks are counted by the second calculating unit 15e for the insertion length detector, the second calculating unit 15e for the insertion length detector may detect the insertion quantity according to a table showing a relationship between the counted marks and the insertion length (shown in FIG. 4F).

Every time when the insertion length detected by the insertion length detector 15 increases or decreases by a predetermined length (for example, 1 mm), the reference point setting unit 16 calculates displacement of a force detected by the force detector 13, and sets, as a reference point, a time point when a change occurs by a predetermined first threshold value (a reference point setting threshold value) (for example, 0.1 N) or more in comparison with displacement until a immediately preceding reference point. The reference point here is a point (a time point for individual force measurement) that becomes a reference for individually measuring the force to be applied based on a summed-up force detected by the force detector 13.

The reference point setting unit 16 sets a time point when the insertion length is 0, as a first reference point. The set reference point is output from the reference point setting unit 16 to the database input/output unit 14, and is stored in the measurement information database 9 by the database input/output unit 14.

The reference point setting unit 16 sets a reference point for calculating each force on each place where the guide wire 2 contacts with the blood vessel 3 based on the summed-up value of the force information detected by the force detector 13 and the information about the insertion length detected by the insertion length detector 15, and the set reference point is output from the reference point setting unit 16 to the database input/output unit 14.

When the operator 6 inserts the guide wire 2, the insertion length detector 15 detects a length along which the guide wire 2 is inserted into the body, using the timer 36 at every certain constant time (for example, every 4 msec), and outputs the lengths as well as the times to the database input/output unit 14, so as to store them in the measurement information database 9.

<<Individual Force Calculating Unit 11>>

The individual force calculating unit 11 calculates forces P1, P2, P3, and P4 to be applied at the respective reference points calculated by the reference point calculating unit 10, respectively, from the summed-up value of the force Pt detected by the force detector 13 based on the information obtained from the force detector 13 and the information from the reference point calculating unit 10 obtained via the database input/output unit 14, so as to output the forces to the database input/output unit 14.

Concretely, the individual force calculating unit 11 divides a value, which is obtained by subtracting information (value) of the force at the immediately preceding reference point from information (value) of the force detected by the force detector 13, by the number of the reference points that have been set, and adds the divided value to individual values at the respective reference points. The individual values calculated by the individual force calculating unit 11 as well as the reference points are output from the individual force calculating unit 11 to the database input/output unit 14.

<<Measurement Information Database 9>>

The database input/output unit 14 stores, in the measurement information database 9, the information about the force detected by the force detector 13 and the insertion length detected by the insertion length detector 15 as well as times using the timer 36. Further, the database input/output unit 14 stores, as a pair, the information about the reference points calculated by the reference point calculating unit 10 and the information about the individual forces at the reference points calculated by the individual force calculating unit 11 into the measurement information database 9. The measurement information is output into and input from the measurement information database 9 by the database input/output unit 14.

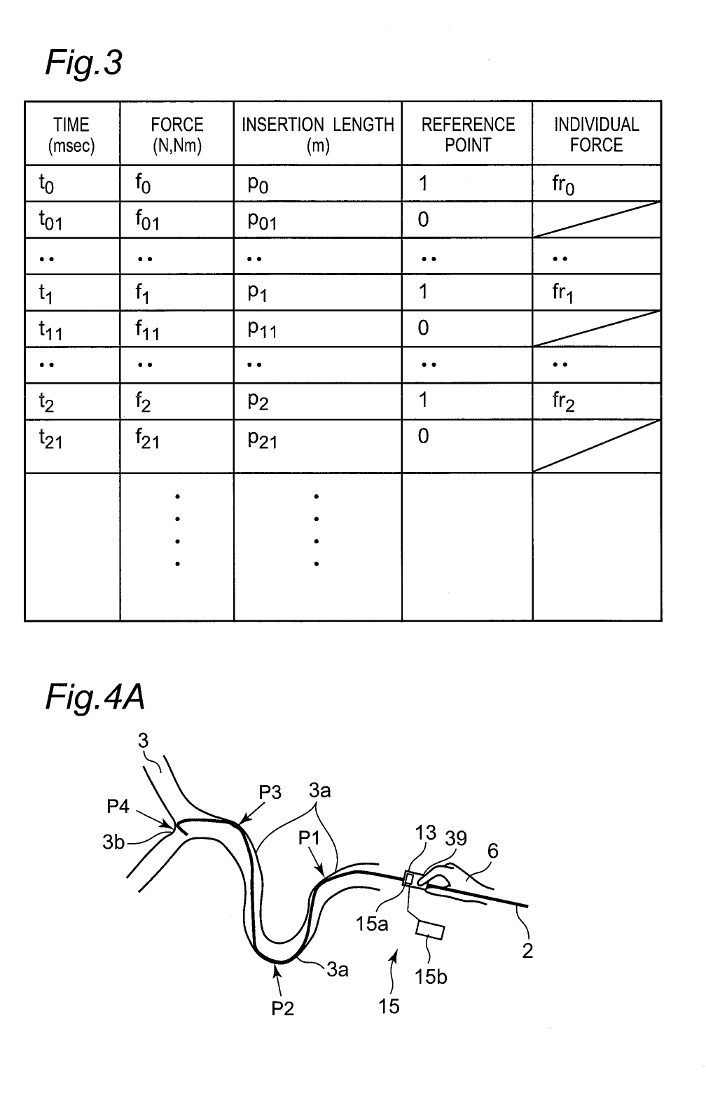

FIG. 3 illustrates one example of information contents of the measurement information database 9.

(1) Column of “TIME” shows information about time when the insertion work is done. In the first embodiment, the time is shown in milliseconds (msec).

(2) Column of “FORCE” shows information about a force detected by the force detector 13. In the first embodiment, the force in the insertion direction is shown in newtons (N), and the force in a rotating direction around the insertion direction is shown in newton meters (Nm).

(3) Column of “INSERTION LENGTH” shows the insertion length of the guide wire 2 detected by the insertion length detector 15. In the first embodiment, the insertion length is shown in meters (m).

(4) Column of “REFERENCE POINT” shows a reference point calculated by the reference point calculating unit 10. When the reference point is set, “1” is set in the corresponding time column, and when no reference point is set, “0” is set.

(5) Column of “INDIVIDUAL FORCE” shows information about a force calculated by the individual force calculating unit 11. In the first embodiment, the force in the insertion direction is shown in newtons (N), and the force in the rotating direction around the insertion direction is shown in newton meters (Nm).

<<Force Deciding Unit 12>>

When the force calculated by the individual force calculating unit 11 is a predetermined second threshold value (load deciding threshold value) (for example, 0.5 N) or more based on the information calculated by the individual force calculating unit 11, the force deciding unit 12 decides that a load is applied to the blood vessel 3. The decided result as well as the force calculated by the individual force calculating unit 11 is output to the decided result notification unit 8.

<<Decided Result Notification Unit 8>>

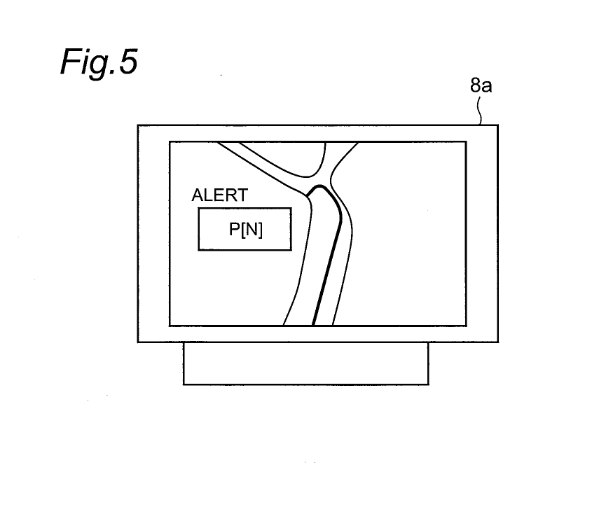

The decided result notification unit 8 is a device that notifies the operator 6 of the result decided by the force deciding unit 12 based on the information from the force deciding unit 12, and is composed of the monitor 8a or the speaker 8b. Concretely, as indicated on the monitor 8a in FIG. 5 as one example of the decided result notification unit 8, the force P [N] detected by the individual force calculating unit 11 as well as an X-ray image imaged by the X-ray imaging device 5 is displayed, and when the force deciding unit 12 decides that a load is applied to the blood vessel 3, a warning such as “ALERT” is displayed. Further, when the force deciding unit 12 decides that a load is applied to the blood vessel 3, a warning sound is sounded from the speaker 8b as another example of the decided result notification unit 8 to warn the operator.

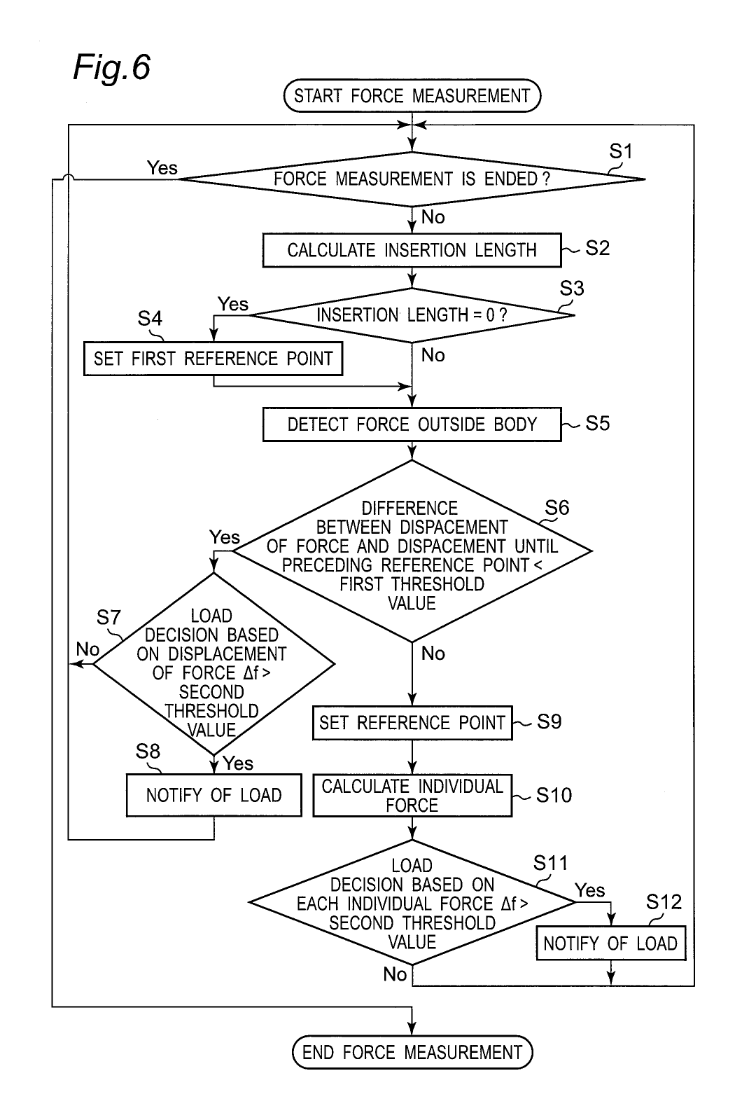

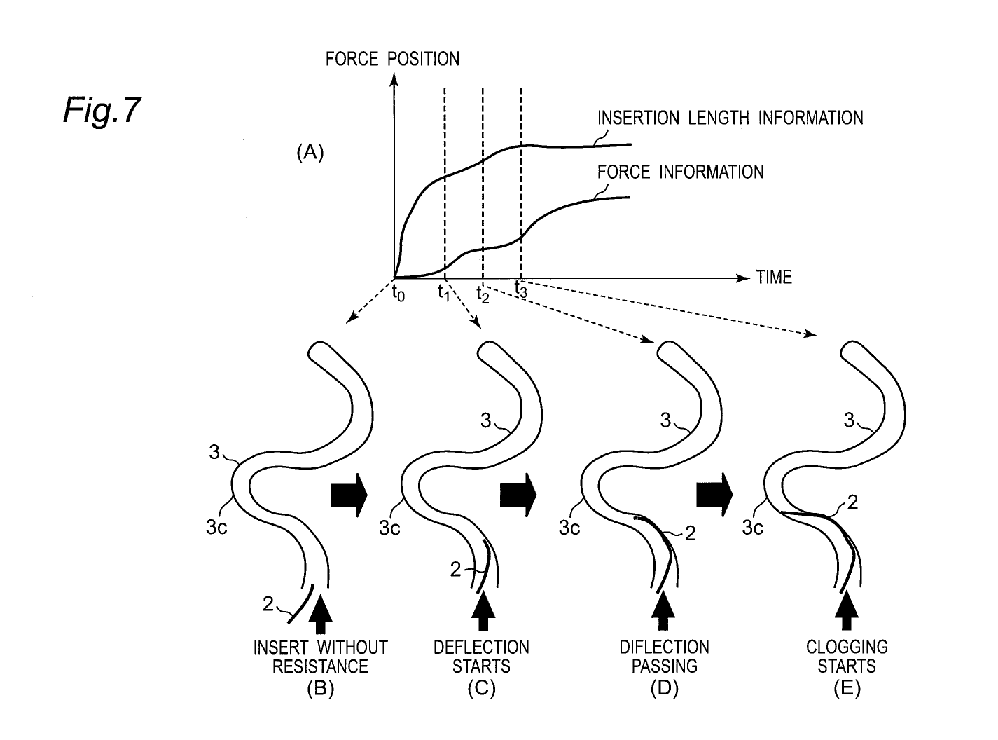

A force measuring step to be executed by the force measurement apparatus 1 according to the first embodiment is described below. FIG. 6 is a flowchart of the force measurement apparatus 1 according to the first embodiment. A work for inserting the guide wire 2 into the blood vessel 3 with meandering portions 3c is described as shown in (B) to (D) FIG. 7.

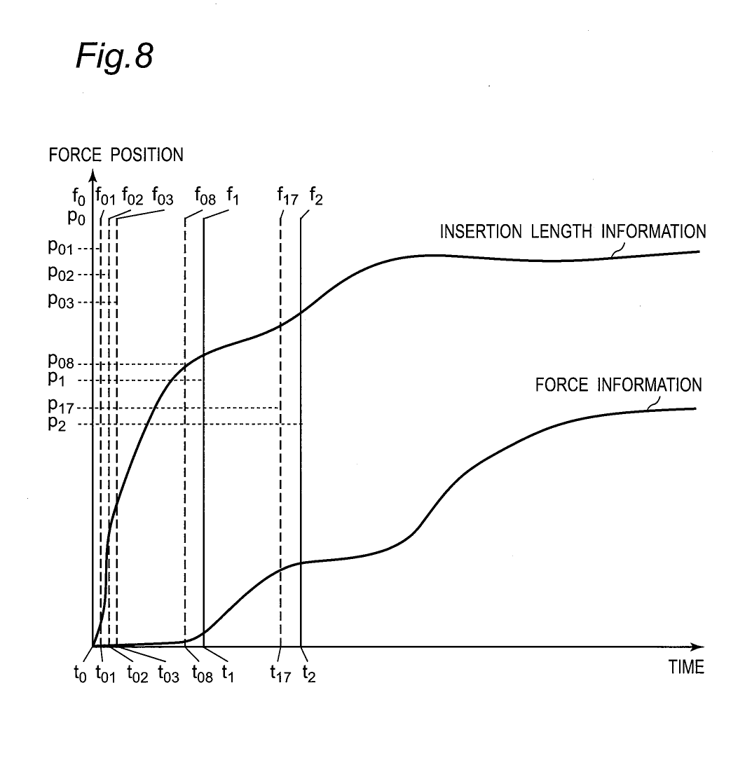

(A) of FIG. 7 and FIG. 8 (FIG. 8 is an enlarged graph of (A) of FIG. 7) are graphs in which the force detected by the force detector 13 and the insertion length detected by the insertion length detector 15 are plotted as the abscissa time during the insertion work shown in (B) to (D) of FIG. 7.

Upon receiving instructions for starting the force measurement from the input/output IF 7, the force measurement controller 200 starts the force measuring process in the force measurement apparatus 1.

Firstly, at step S1, the force measurement controller 200 decides whether the input/output IF 7 issues a command for ending the force measurement. When the decision is made that the input/output IF 7 issues the command for ending the force measurement, the force measurement controller 200 ends the force measuring process in the force measurement apparatus 1. When the decision is made that the input/output IF 7 does not issue the command for ending the force measurement, the force measurement controller 200 allows the force measuring process to go to next step S2.

At step S2, the insertion length detector 15 detects the insertion length along which the guide wire 2 is inserted into the blood vessel 3.

Next, at step S3, the reference point setting unit 16 decides whether the insertion length is “0” based on the detected result in the insertion length detector 15. When the reference point setting unit 16 decides that the insertion length detected by the insertion length detector 15 is “0”, the force measuring process goes to step S4. When the reference point setting unit 16 decides that the insertion length detected by the insertion length detector 15 is not “0”, the force measuring process goes to step S5.

When the reference point setting unit 16 decides at step S4 that the insertion length detected by the insertion length detector 15 is “0”, this means the time point when the insertion is started as shown in (B) of FIG. 7, and the reference point setting unit 16 sets this time point as a first reference point (see time point “t₀” in (A) of FIG. 7). Further, the reference point set by the reference point setting unit 16 is output to the database input/output unit 14, and is stored in the measurement information database 9 (the column of the reference point at a time point t₀ in FIG. 3 indicates “1”). Thereafter, the force measuring process goes to step S5.

At step S5, the force detector 13 detects a force to be applied to the guide wire 2 from the outside of the body. The force detected by the force detector 13 as well as the time is output to the database input/output unit 14 using the timer 36, and the force and the time are stored in the measurement information database 9. The force detected by the force detector 13 is measured by the force detector 13 in a manner that forces at the meandering portions 3c or the branch portions of the blood vessel 3 are added up as described above. Therefore, the reference point is calculated after step S6, and the individual forces at the respective reference points are calculated by the individual force calculating unit 11 so that the forces at the meandering portions 3c are calculated by the individual force calculating unit 11.

Next, at step S6, a next reference point is calculated by the reference point calculating unit 10 composed of the insertion length detector 15 and the reference point setting unit 16. Every time when the insertion length detector 15 detects that the insertion length increases/decreases by a predetermined length (for example, 1 mm), displacement of the force detected by the force detector 13 is calculated by the reference point setting unit 16. Concretely, the reference point setting unit 16 calculates displacement Δf₀₁=f₀₁−f₀ of the force at a time point t₀₁ when the insertion length increases by the predetermined length (pₛ=p₀₁−p₀) in FIG. 8. Symbol f₀₁ represents the force at the time point t₀₁, and symbol f₀ represents the force at the time point t₀. Thereafter, a correspondence relationship between the forces and the time points are similar. The reference point setting unit 16 decides whether the displacement Δf₀₁ of the force at the time point t₀₁ changes by a predetermined first threshold value (for example, 0.1 N) or more in comparison with displacement until the immediately preceding reference point (step S6). When the immediately preceding reference point (the reference point at the time point t₀) is the first reference point like an example of FIG. 8, the reference point setting unit 16 decides whether the displacement Δf₀₁ of the force is the predetermined first threshold value (for example, 0.1 N) or more. In the example of FIG. 8, the reference point setting unit 16 decides that the displacement Δf₀₁ of the force is less than the predetermined first threshold value (for example, 0.1 N), and the reference point setting unit 16 does not set the time point t₀₁ as a next reference point. When the reference point setting unit 16 does not set the reference point, the force measuring process goes to step S7. When the reference point setting unit 16 sets the reference point, the force measuring process goes to step S9.

Since the reference point setting unit 16 does not set the reference point at step S7, the decision in the force deciding unit 12 is urged via the database input/output unit 14. As a result, the force deciding unit 12 decides whether the displacement Δf₀₁ of the force is the predetermined second threshold value (for example, 0.5 N) or more. When the displacement Δf₀₁ of the force is the predetermined second threshold value or more at step S7, the force measuring process goes to step S8.

At step S8, the monitor 8a or the speaker 8b of the decided result notification unit 8 notifies the operator of a warning based on the decision in the force deciding unit 12. Thereafter, the force measuring process returns to step S1.

Every time when the insertion length increases by the predetermined length at step S6, the displacement of the force detected by the force detector 13 is compared, but as shown in (E) of FIG. 7, for example, the distal end of the guide wire 2 contacts with the blood vessel 3 to be clogged, and even if the guide wire 2 is pushed towards the blood vessel 3 from the outside of the body, the insertion quantity of the guide wire 2 does not change in some cases. In such a case where the insertion length does not change for predetermined time or more, for example, the displacement of the force detected by the force detector 13 is not compared by the reference point setting unit 16 every time when the insertion length increases or decreases by the predetermined length, but the displacement of the force detected by the force detector 13 is compared by the reference point setting unit 16 every time when the predetermined time passes.

When the displacement Δf₀₁ of the force is not the predetermined second threshold value (for example, 0.5 N) or more at step S7, the force measuring process returns to step S1 and after step S2, step S3, and step S5, the reference point calculation is started similarly. At step S6, displacement Δf₀₂=f₀₂−f₀₁ of the force P₀₂ at a time point t₀₂ when the force is increased from P₀₁ by a predetermined length (force Pₛ) in FIG. 8 is calculated by the reference point setting unit 16. The reference point setting unit 16 decides whether the displacement Δf₀₂ of the force changes by the predetermined first threshold value (for example, 0.1 N) or more in comparison with the displacement until the immediately preceding reference point. In the example of FIG. 8, the displacement Δf₀₂ of the force is less than the predetermined first threshold value, and the time point t₀₂ is not set as a next reference point by the reference point setting unit 16. At this time, similarly to the above case, the force measuring process goes through step S7 and step S8 and returns to step S1 and goes through step S2, step S3, and step S5 so as to start the reference point calculation similarly. The reference point setting unit 16 calculates sequentially as to whether a reference point can be set at time points t₀₃, t₀₄, . . . , t₀₇. In the example of FIG. 8, the reference point cannot be set up to a time point t₀₈ by the reference point setting unit 16. Then, the reference point setting unit 16 calculates displacement Δf₁₀=f₁−f₀₈ of the force at the time point t₁ when the insertion length increases by the predetermined length (pₛ=p₁−p₀₈). The reference point setting unit 16 decides whether the displacement Δf₁₀ of the force changes by the predetermined first threshold value (for example, 0.1 N) or more in comparison with the displacement up to the immediately preceding reference point (step S6). In the example of FIG. 8, when the reference point setting unit 16 decides that the displacement Δf₁₀ of the forces from the time point t₀₈ to the time point t₁ is the predetermined first threshold value (for example, 0.1 N) or more, the force measuring process goes to step S9.

At step S9, the reference point setting unit 16 sets the time point t₁ as a next reference point. The reference point set by the reference point setting unit 16 is output to the database input/output unit 14, described later, and is stored in the measurement information database 9 (the column of the reference point at the time point t₁ indicates “l” in FIG. 3). At this time, as shown in (C) of FIG. 7, at the reference point of the time point t₁, the guide wire 2 contacts with the wall of the blood vessel 3 so as to start to be deflected.

Next, at step S10, the individual force calculating unit 11 calculates individual forces at the respective reference points. The individual force calculating unit 11 divides a value, which is obtained by subtracting the information about the force at the immediately preceding reference point from the information about the force detected by the force detector 13, by the number of the reference points that have been set, and adds the obtained value to the individual forces at the respective reference points so as to calculate the individual forces at the respective reference points. When the individual forces at the respective references points are a predetermined third threshold value (for example, 0.01 N) or less in the individual force calculating unit 11, these reference points are not counted as the number of the reference points, and the calculated force is not added to these uncounted reference points. Concretely, the individual force at the reference point of the time point t₁ in FIG. 8 is described as an example. A value Δf₁ (=f₁−f₀), which is obtained by subtracting a force t₀ at a immediately preceding reference point t₀ from the force f₁ at the reference point of the time point t₁, is divided by the number of the reference points that have been set (“2” that is the number of the reference points of the time points t₀ and t₁ in this example, but since the force f₀ at the reference point of the time point t₀ is the third threshold value or less, the number of the reference values is “1”). The divided value is set as the individual force at the reference point of the time point t₁. Since the force f₀ at the reference point of the time point t₀ is the third threshold value or less, the force obtained by divided by the number of the reference points is not added. That is to say, in this example, the individual force at the reference point of the time point t₁ is such that fᵣ₁=Δf₁/1. The individual force fᵣ₀ at the first reference point t₀ becomes the force f₀ at the reference point of the time point t₀. The individual force calculated by the individual force calculating unit 11 is output from the individual force calculating unit 11 to the database input/output unit 14, and is stored in the measurement information database 9 (in this example, individual forces fᵣ₀ and fᵣ₁ at the reference points of the time points t₀ and t₁ in FIG. 3 are stored).

Next, at step S11, the force deciding unit 12 decides a load for the individual forces calculated by the individual force calculating unit 11. Concretely, the force deciding unit 12 decides whether each of the individual force fᵣ₀ at the reference point of the time point t₀ obtained before and the individual force fᵣ₁ at the reference point of the time point t₁ is the second threshold value (for example, 0.5 N) or more. When the force deciding unit 12 decides at step S11 that any one of the forces is the second threshold value or more, the force measuring process goes to step S12.

At step S12, the monitor 8a or the speaker 8b of the decided result notification unit 8 notifies the operator of a warning based on the decision in the force deciding unit 12.

When the force deciding unit 12 decides at step S11 that any one of the forces is not the second threshold value (for example, 0.5 N) or more, the force measuring process returns to step S1 so that a next reference point is calculated.

The first threshold value, the second threshold value, or the third threshold value varies according to types (blood vessel diameter or portion) or a state of the blood vessel 3 of a patient (the human body 4), and for example, the operator can select the value from a plurality of threshold values generated in advance, or the operator can input the value to the reference point setting unit 16, the force deciding unit 12, or the individual force calculating unit 11 using an input device such as a keyboard or a button.

Next, the calculation of a reference point t₂ after the reference points t₀ and t₁ in the reference point calculating unit 10 is described as an example with reference to FIG. 8. With return to step S1, the sequence again goes through step S2, step S3, and step S5, and the reference point calculating unit 10 starts the calculation of the reference points. The reference point setting unit 16 makes a calculation whether reference points can be set sequentially. Suppose that the reference point setting unit 16 cannot set reference points until at the time point t₁₇ in the example of FIG. 8. The reference point setting unit 16 calculates displacement Δf₂₀=f₂−f₁₇ of the force at the time point t₂ when the insertion length increases by a predetermined length (for example, 1 mm) (pₛ=p₂−p₁₇). The reference point setting unit 16 decides whether the displacement Δf₂₀ of the force changes by the predetermined first threshold value (for example, 0.1 N) or more in comparison with the displacement until the immediately preceding reference point (step S6). In this example, since the immediately preceding reference point is the time point t₁, the reference point setting unit 16 decides whether an absolute value of a difference between the displacement Δf₁₀=f₁−f₀₈ of the force at the reference point of the time point t₁ and the displacement Δf₂₀ of the force is the predetermined first threshold value or more (step S6). In the example of FIG. 8, the reference point setting unit 16 decides that the absolute value of the difference between the displacement Δf₁₀ of the force and the displacement Δf₂₀ of the force is the predetermined first threshold value or more, and sets the time point t₂ as a next reference point (step S9). The reference point set by the reference point setting unit 16 is output from the reference point setting unit 16 to the database input/output unit 14, and is stored in the measurement information database 9 (“1” is set in the column of reference point at the time point t₂ in FIG. 3). As shown in (D) of FIG. 7, at the reference point of the time point t₂, the guide wire 2 contacts with the blood vessel 3 so as to be further deflected, and passes through the meandering portions 3c.

Next, at step S10, the individual force calculating unit 11 calculates individual forces at the respective reference points. As described above, the individual force calculating unit 11 divides the value, which is obtained by subtracting the information about the force at the immediately preceding reference point from the information about the force detected by the force detector 13, by the number of the reference points that have been set, and adds the obtained value as the result to the individual forces at the respective reference points, so as to calculate the individual forces at the respective reference points. The individual forces at the reference points of the time points t₁ and t₂ in FIG. 8 are described as an example. A value Δf₂ (=f₂−f₁), which is obtained by subtracting the force f₁ at the immediately preceding reference point t₁ from the force f₂ at the reference point of the time point t₂, is divided by the number of the reference points that have been set (in this example, besides the reference point of the time point t₀, the reference points are the reference points of the time points t₁ and t₂, and thus the number of the reference points is “2”). This divided value is set as the individual force at the reference point t₂. In this example, an individual force fᵣ₂ at the reference point of the time point t₂ is such that fᵣ₂=Δf₂/2. An individual force fᵣ₀ at the first reference point t₀ becomes the force f₀ at the reference point of the time point t₀. Further, an individual force f_{r1(new)} at the reference point t₁ is a value obtained by adding Δf₂/2 to the individual force (f_{r1(old)}) calculated before, namely, f_{r1(new)}=f_{r1(old)}+Δf₂/2. The individual force calculated by the individual force calculating unit 11 in such a manner is output from the individual force calculating unit 11 to the database input/output unit 14, and is stored in the measurement information database 9 (in this example, the individual forces fᵣ₀, fᵣ₁, and fᵣ₂ are stored at the reference points of the time points t₀, t₁, and t₂ in FIG. 3).

Next, at step S11, the force deciding unit 12 decides a load for individual forces calculated by the individual force calculating unit 11. Concretely, the force deciding unit 12 decides whether each of the individual force fᵣ₀ at the reference point t₀ obtained before, the individual force fᵣ₁ at the reference point t₁, and the individual force fᵣ₂ at the reference point t₂ is the second threshold value (for example, 0.5 N) or more (step S11). When the force deciding unit 12 decides at step S11 that even one of the three individual forces is the second threshold value (for example, 0.5 N) or more, the monitor 8a or the speaker 8b of the decided result notification unit 8 notifies the operator of a warning (step S12). When the force deciding unit 12 decides at step S11 that all the three individual forces are not the second threshold value or more, the sequence returns to step S1, and a next reference point is calculated.

<<Effect of the First Embodiment>>

The reference point calculating unit 10 calculates a time point when the displacement of the force detected by the force detector 13 changes by the predetermined threshold value or more, that is, when the operator makes the guide wire 2 contact with the blood vessel 3 or makes the guide wire 2 pass through the meandering portion. Further, the individual force calculating unit 11 distributes the summed-up force at the operator's hand detected by the force detector 13 to the forces at the respective reference points based on the reference points calculated by the reference point calculating unit 10, so as to be capable of estimating individual loads on the blood vessel 3 at, for example, the time points when the guide wire 2 contacts with the blood vessel 3 or passes through the meandering portion. Further, the force deciding unit 12 decides the load for the forces calculated individually, so as to be capable of detecting the loads with a constant threshold value regardless of the number of the meandering portions.

### Second Embodiment

A force measurement apparatus 1B according to the second embodiment of the present invention is described below. The second embodiment is described by using the force measuring motion when a guide wire 2 is inserted into a blood vessel 3 as an example similarly to the first embodiment as shown in FIG. 1.

Since the basic constitutions of a measurement information database 9, a database input/output unit 14, a force detector 13, a force deciding unit 12, and a decided result notification unit 8 in the second embodiment of the present invention are similar to those in the first embodiment, description about common portions is omitted, and only different portions are described in detail below.

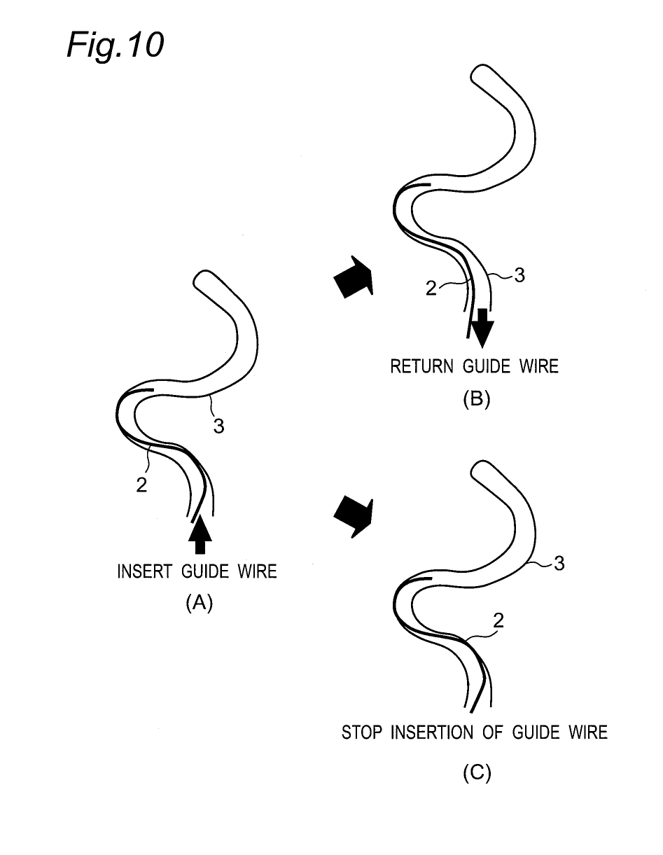

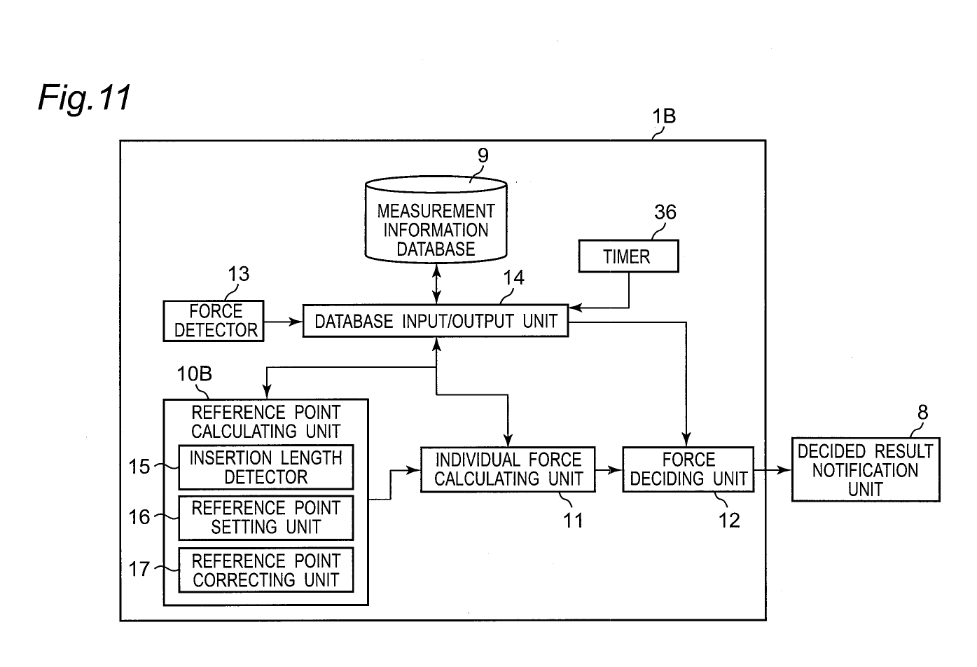

The first embodiment describes the work for inserting the guide wire 2 into the blood vessel 3, but the second embodiment describes the force measurement apparatus 1B in a case where the guide wire 2, which is inserted into the blood vessel 3 as shown in (A) of FIG. 10, is pulled out of the blood vessel 3 in the body as shown in (B) of FIG. 10, and the insertion of the guide wire 2 is stopped as shown in (C) of FIG. 10. FIG. 11 is a constitutional view illustrating the force measurement apparatus 1B according to the second embodiment.

<<Reference Point Calculating Unit 10B>>

The reference point calculating unit 10B is composed of an insertion length detector 15, a reference point setting unit 16, and a reference point correcting unit 17 that functions as one example of a correcting unit. The operations of the insertion length detector 15 and the reference point setting unit 16 are basically similar to those in the first embodiment. When the guide wire 2 is reinserted into the blood vessel 3 after the guide wire 2 is inserted into the blood vessel 3 and is once pulled back partially, the reference point correcting unit 17 makes a correction so that the reference points set by the reference point setting unit 16 between a pulling-back start time point and a reinsertion time point are deleted.

Concretely, the following operation is performed.

As shown in (A) of FIG. 10, since the operation of the insertion length detector 15 at time when the operator carries out the insertion is similar to that in the first embodiment, description thereof is omitted. Further, as shown in (C) of FIG. 10, also in the operation of the insertion length detector 15 at time when the operator stops the insertion, the reference points are calculated using the similar method to the first embodiment.

On the other hand, as shown in (B) of FIG. 10, when the guide wire 2 is pulled out of the blood vessel 3 in the body and the insertion length detected by the insertion length detector 15 decreases to be smaller than the insertion length detected at the immediately preceding time by a predetermined length, the reference point setting unit 16 calculates the reference points in the similar method to the first embodiment. When the reference point setting unit 16 sets the reference points, the reference points are output from the reference point setting unit 16 to the database input/output unit 14, and “2” is stored for the reference point in the measurement information database 9. When the guide wire 2 is inserted into the blood vessel 3 in the body and the insertion length detected by the insertion length detector 15 increases, the reference point setting unit 16 makes a calculation with the similar method to the first embodiment. When the reference point setting unit 16 sets a reference point, the reference point is output from the reference point setting unit 16 to the database input/output unit 14 and “1” is stored for the reference point in the measurement information database 9.

The reference point correcting unit 17 decides whether the reference point set by the reference point setting unit 16 is a reference point after the next to the immediately preceding reference point with “2”. That is to say, the reference point correcting unit 17 detects whether the reference point is a reference point next to a time point when the pulling-back of the guide wire 2 is ended and the insertion of the guide wire 2 is restarted.

When the reference point correcting unit 17 decides that the reference point set by the reference point setting unit 16 is the reference point after the next to the immediately preceding reference point with “2”, the reference point correcting unit 17 corrects the reference points that have been set until that time. This correction is for, for example, deleting reference points present between the start of the pulling-back and the end of the pulling-back. When the reference point correcting unit 17 decides that the reference point set by the reference point setting unit 16 is not the reference point after the next to the immediately preceding reference point with “2”, an individual force calculating unit 11 calculates an individual force similarly to the first embodiment as described later.

The reference point correcting unit 17 searches for a reference point whose insertion quantity becomes equal to or more than an insertion quantity at a time point when the pulling-back of the guide wire 2 is ended and the insertion of the guide wire 2 is restarted, sequentially starting from the first reference point. The reference point correcting unit 17 sequentially corrects “1” for reference points after the searched reference point into “−1”, and the correction is ended at a time point when the correction into “−2” is made for the reference point with “2”.

<<Individual Force Calculating Unit 11>>

The individual force calculating unit 11 calculates forces (individual forces) to be applied at the reference points calculated by the reference point calculating unit 10B based on the summed-up value of the forces detected by the force detector 13, and outputs the calculated individual forces to the database input/output unit 14 so as to store them in the measurement information database 9. Concretely, the individual force calculating unit 11 makes a calculation in a manner that the information about the force at the immediately preceding reference point is subtracted from the information about the forces detected by the force detector 13, the subtracted value is divided by the number of the reference points that have been set, and the divided value is added to the individual forces at the respective reference points. The number of the reference points is counted by the individual force calculating unit 11 in a manner that the reference points that are “−1”, “−2”, and “0” are reduced. The individual forces calculated by the individual force calculating unit 11 as well as the reference points are output to the database input/output unit 14 so as to be stored in the measurement information database 9.

(Force Measuring Step)

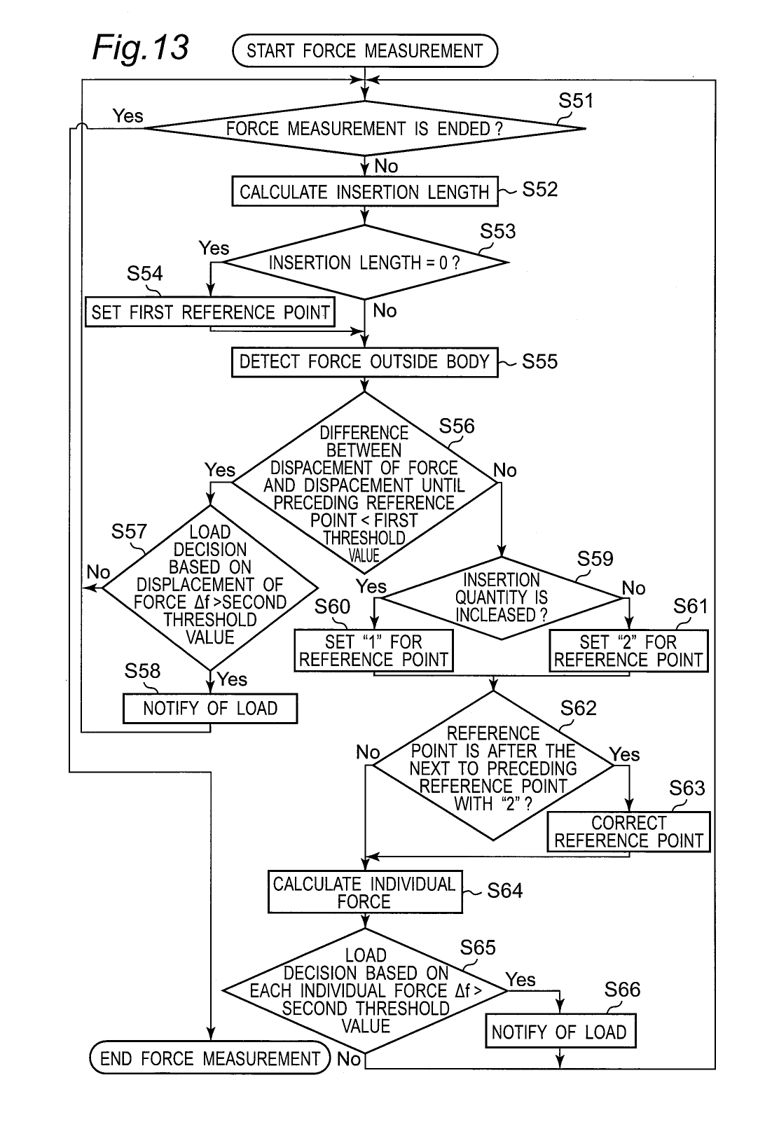

The force measuring step in the force measurement apparatus 1B according to the second embodiment is described with reference to a flowchart in FIG. 13.

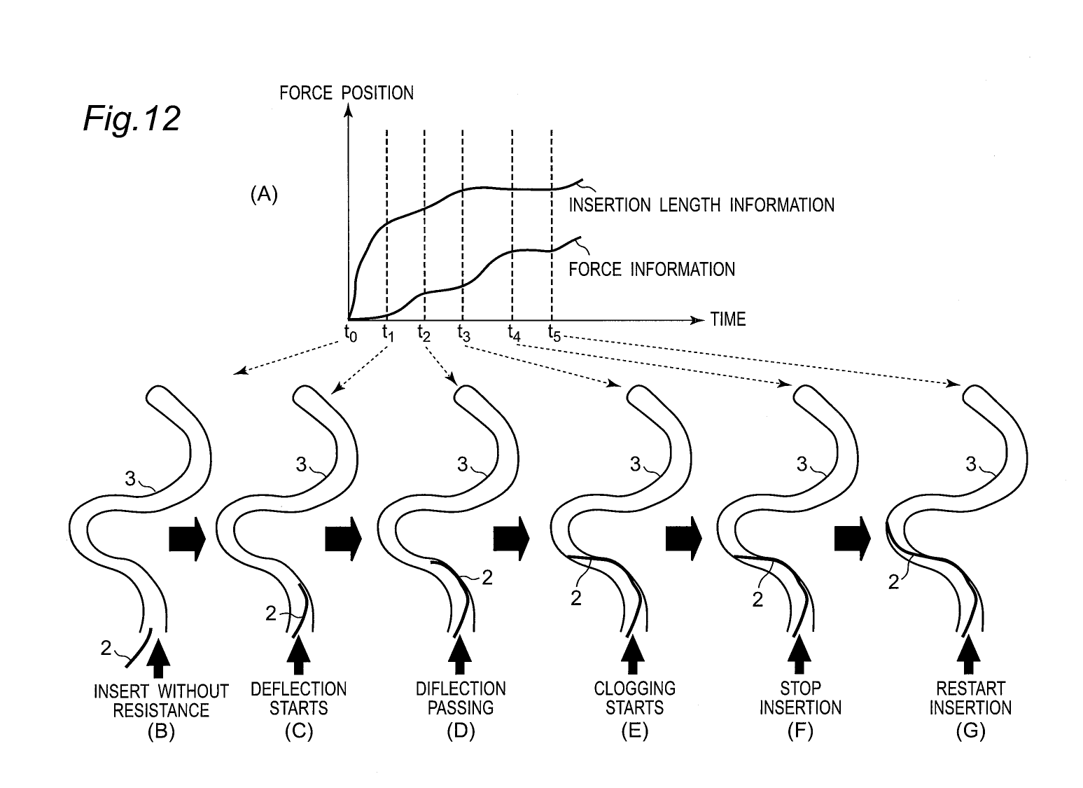

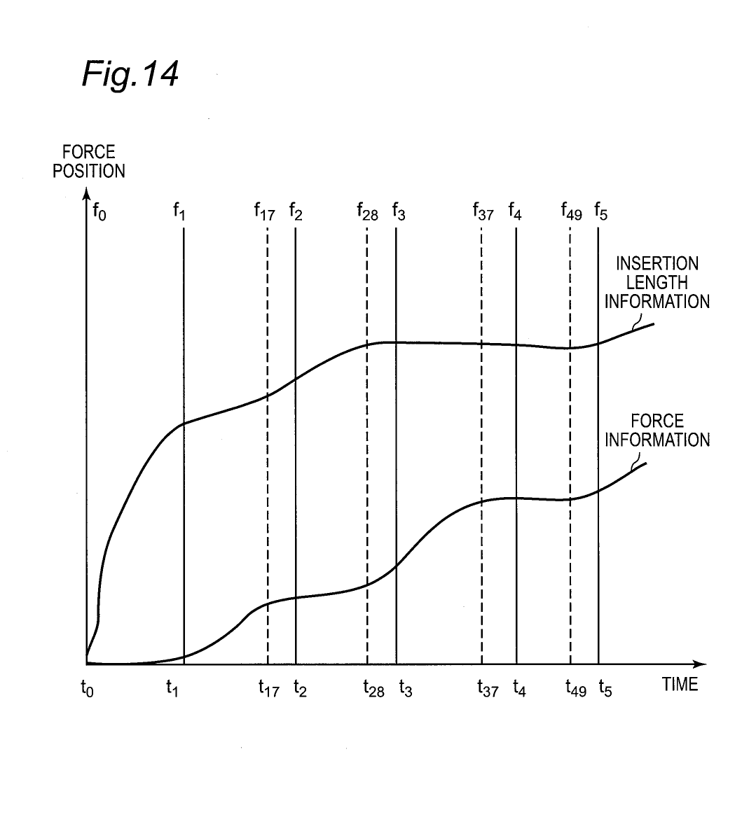

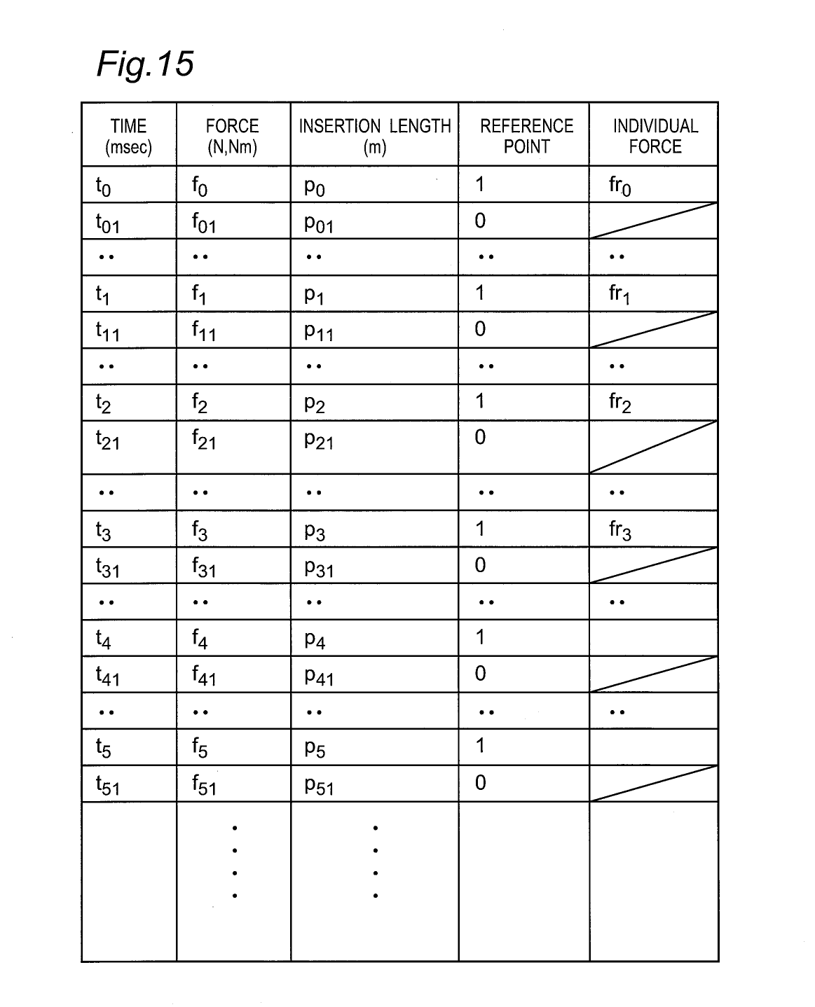

A case where the insertion of the guide wire 2 is stopped after a time point t₃ in (A) of FIG. 7 in the first embodiment is described first. When the insertion is stopped, the flowchart of FIG. 13 in the first embodiment is used. (A) of FIG. 12 is a graph where the insertion lengths and the forces are plotted when the insertion is restarted after the insertion is stopped after the time point t₃ in (A) of FIG. 7. FIG. 14 is an enlarged graph of (A) of FIG. 12. Further, the measurement information database 9 according to the second embodiment is shown in FIG. 15.

<<<Calculation of Reference Points of Time Points t₃ and t₄>>

Also in the second embodiment, it is assumed that the reference points of the first time point t₀ to the time point t₂ are set by the similar method to the first embodiment, namely, at step S1 to step S12 in FIG. 6, and the calculation of the reference points of the time points t₃ and t₄ is described below.

Similarly to the first embodiment, upon receiving the command for starting the force measurement from an input/output IF 7, a force measurement controller 200 starts the force measuring process in the force measurement apparatus 1B.

Firstly, at step S1 in FIG. 6, the force measurement controller 200 decides whether the input/output IF 7 issues the command for ending the force measurement. When the decision is made that the input/output IF 7 issues the command for ending the force measurement, the force measurement controller 200 ends the force measuring process in the force measurement apparatus 1B. When the decision is made that the input/output IF 7 does not issue the command for ending the force measurement, the force measurement controller 200 allows the force measuring process to go to next step S2.

At step S2, the insertion length detector 15 detects the insertion length along which the guide wire 2 is inserted into the blood vessel 3.

Next, at step S3, the reference point setting unit 16 decides whether the insertion length is “0” based on the detected result in the insertion length detector 15. When the reference point setting unit 16 decides that the insertion length detected by the insertion length detector 15 is “0”, the force measuring process goes to step S4. When the reference point setting unit 16 decides that the insertion length detected by the insertion length detector 15 is not “0”, the force measuring process goes to step S5.

When the reference point setting unit 16 decides at step S4 that the insertion length detected by the insertion length detector 15 is “0”, it means the time point at which the insertion is started as shown in (B) of FIG. 12, and the reference point setting unit 16 sets that time point as the first reference point (see the time point “t₀” in (A) of FIG. 12). Further, the reference point set by the reference point setting unit 16 is output to the database input/output unit 14 and is stored in the measurement information database 9 (“1” is set in the column of reference point for the time point t₀ in FIG. 15). Thereafter, the force measuring process goes to step S5.

At step S5, the force detector 13 detects a force to be applied to the guide wire 2 from the outside of the body. The values detected by the force detector 13 as well as the times using a timer 36 are output to the database input/output unit 14, and are stored in the measurement information database 9.

Next, at step S6, the reference point calculating unit 10B calculates reference points after the time point t₃. In the first embodiment, every time when the insertion length increases or decreased by the predetermined length, the reference point setting unit 16 calculates the displacement of a force detected by the force detector 13. As described also in the first embodiment, when the insertion length does not change for predetermined or more time, the displacement of the forces detected by the force detector 13 is not compared by the reference point setting unit 16 every time when the insertion length increases by the predetermined length, but the displacement of the forces detected by the force detector 13 is compared by the reference point setting unit 16 every time when the predetermined time elapses. Even when the predetermined time elapses, the insertion length does not change after the time point t₃ in FIG. 14, and thus the reference point setting unit 16 calculates displacement of forces Δf₃₀=f₃−f₂₈ at a time point t₂₈ after the elapse of the predetermined time and the time point t₃. The reference point setting unit 16 compares the displacement Δf₃₀ of the forces with the displacement of the forces until the immediately preceding reference point, and the reference point setting unit 16 decides whether the displacement changes by a predetermined first threshold value or more (step S6). In an example of FIG. 14, the reference point setting unit 16 decides that an absolute value of a difference between displacement Δf₂₀ of the force at the immediately preceding reference point and the displacement Δf₃₀ of the force is the predetermined first threshold value or more, the force measuring process goes to step S9. The reference point setting unit 16 compares the displacement Δf₃₀ of the force with the displacement of the forces until the immediately preceding reference points, and when the reference point setting unit 16 decides as being less than the predetermined first threshold value, the force measuring process goes to step S7.

At step S9, the reference point setting unit 16 sets the time point t₄ as a next reference point. That is to say, the reference points set by the reference point setting unit 16 are output from the reference point setting unit 16 to the database input/output unit 14, and are stored in the measurement information database 9 (“1” is set in the column of reference point for the time point t₄ FIG. 15).

Next, similarly to the first embodiment, the individual force calculating unit 11 calculates individual forces at the respective reference points at step S10, and the calculated individual forces are output from the individual force calculating unit 11 to the database input/output unit 14 so as to be stored in the measurement information database 9 (in this example, the individual forces fᵣ₀, fᵣ₁, fᵣ₂, and fᵣ₃ are stored at the time points t₀, t₁, t₂, and t₃ in FIG. 15).

Next, similarly to the first embodiment, the force deciding unit 12 decides a load for the individual forces calculated by the individual force calculating unit 11 at step S11. Concretely, the force deciding unit 12 decides whether the individual forces at the respective reference points are a second threshold value (for example, 0.5 N) or more. When the decision is made at step S11 that even one of the individual forces is the second threshold value or more, a monitor 8a or a speaker 8b of the decided result notification unit 8 notifies the operator of a warning (step S12). Thereafter, the force measuring process returns to step S1, and calculates a next reference point. When the force deciding unit 12 decides at step S11 that all the individual forces are not the second threshold value or more, the force measuring process returns to step S1, and a next reference point is calculated.

<<Insertion is Stopped at Time Point t₄>>

Next, the motion for stopping the insertion at the time point t₄ is described.

In the first embodiment, the reference point setting unit 16 calculates the displacement of forces detected by the force detector 13 at step S6 at every time when the insertion length increases or decreases by a predetermined length. As described also in the first embodiment, however, when the reference point setting unit 16 decides that the insertion length does not change for predetermined time (for example, 1 sec) or more, the reference point setting unit 16 does not compare the displacement of the forces detected by the force detector 13 at every time the insertion length increases by the predetermined length, but the reference point setting unit 16 compares the displacement of the forces detected by the force detector 13 at every time when predetermined time elapses. Even when the predetermined time elapses, the insertion length does not change after the time point t₄ in FIG. 14, and thus the reference point setting unit 16 calculates displacement of forces Δf₄₀=f₄−f₃₇ at a time point t₃₇ after the elapse of the predetermined time and the time point t₃. The reference point setting unit 16 compares the displacement Δf₄₀ with the displacement of the forces until the immediately preceding reference point, and the reference point setting unit 16 decides whether the displacement changes by the predetermined first threshold value or more (step S6). In the example of FIG. 14, when the reference point setting unit 16 decides that the absolute value of the difference between the displacement Δf₃₀ of the forces and the displacement Δf₄₀ of the forces at the immediately preceding reference point is the predetermined first threshold value or more, the reference point setting unit 16 sets the time point t₄ as a next reference point (step S9). The reference points set by the reference point setting unit 16 are output from the reference point setting unit 16 to the database input/output unit 14, and are stored in the measurement information database 9 (“1” is set in the column of reference point for the time point t₄ in FIG. 15).

Next, similarly to the first embodiment, the individual force calculating unit 11 calculates individual forces at the respective reference points at step S10, and the calculated individual forces are output from the individual force calculating unit 11 to the database input/output unit 14 so as to be stored in the measurement information database 9 (in this example, the individual forces fᵣ₀, fᵣ₁, fᵣ₂, fᵣ₃, and fᵣ₄ are stored at the time points t₀, t₁, t₂, t₃, and t₄ in FIG. 15).

Next, similarly to the first embodiment, the force deciding unit 12 decides a load for the individual forces calculated by the individual force calculating unit 11 at step S11. Step S12 is also similar to the first embodiment.

<<Restart of the Insertion at Time Point t₅>>

Next, these motion for restarting the insertion at the time point t₅ is described below.

When the insertion is restarted at the time point t₅, the individual forces at the reference points that have been set do not greatly change. For this reason, the individual force calculating unit 11 calculates an individual force at the time point t₅ using the reference points that have been set. Since the calculating method in the individual force calculating unit 11 is similar to one up to at the time point t₄, description thereof is omitted. Calculated measurement data is shown in FIG. 15.

<<Case where the Guide Wire 2 is Pulled Back>>

Next, the case where the guide wire 2 is pulled back as shown in (B) of FIG. 10 is described as an example.

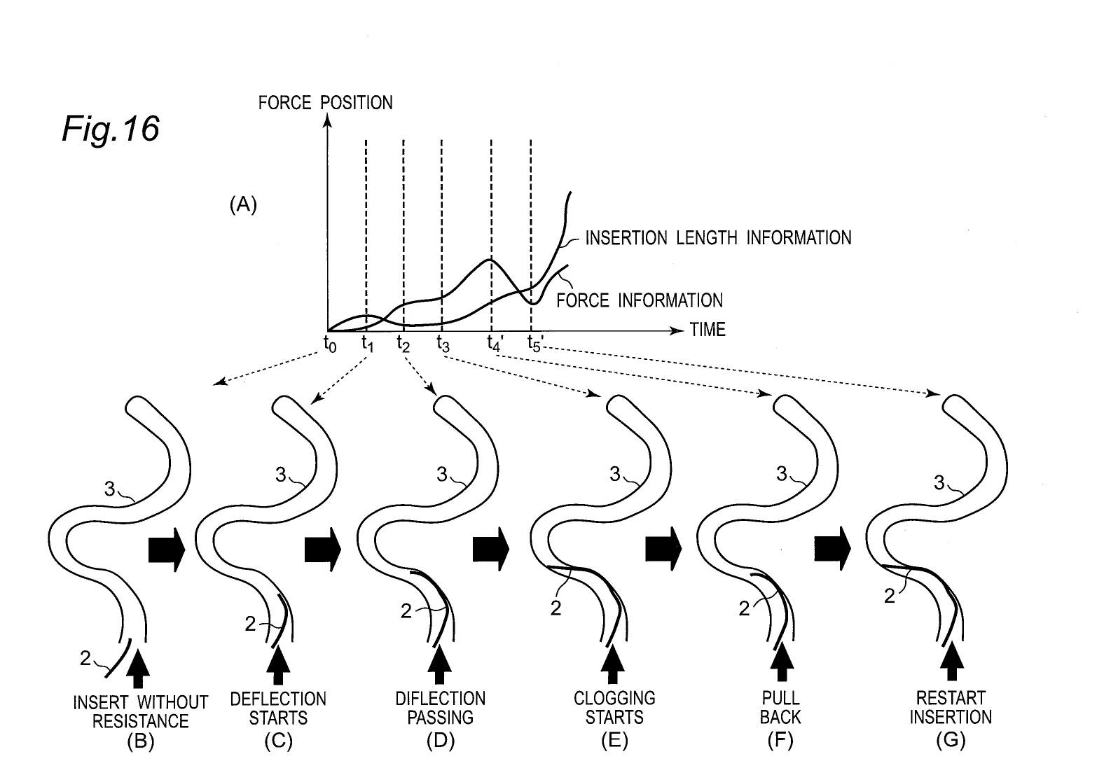

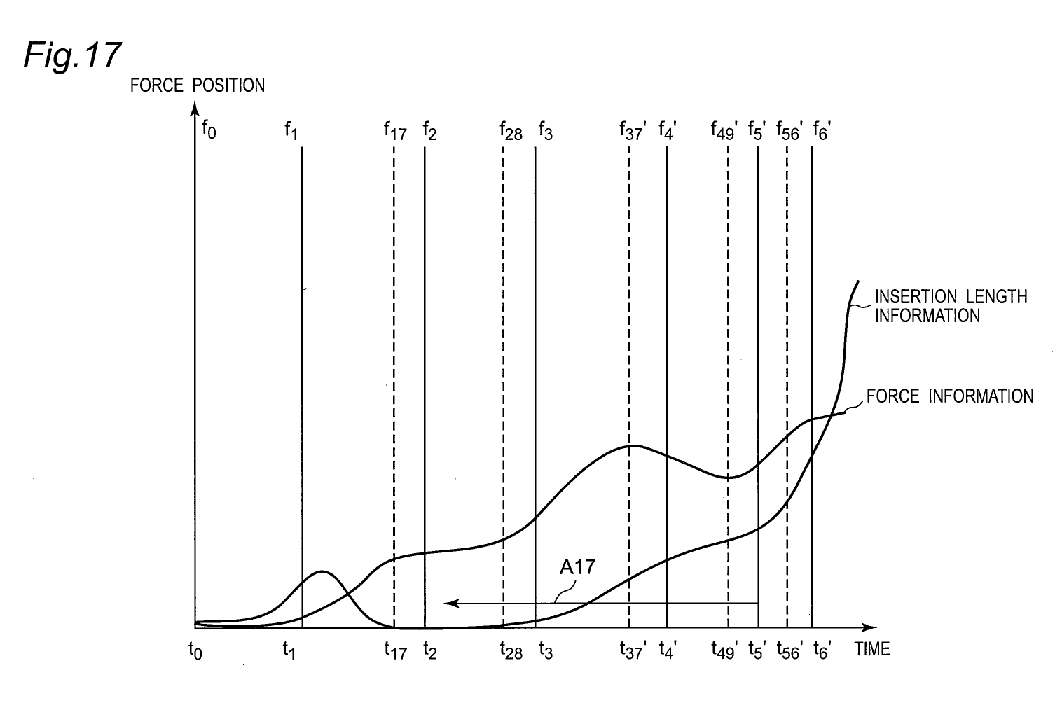

FIG. 13 is a flowchart of the force measurement apparatus 1B according to the second embodiment. (A) of FIG. 16 is a graph of the insertion quantities and the forces at time of the pulling-back and the restart of the insertion, and FIG. 17 is an enlarged graph of (A) of FIG. 16.

In FIG. 17, supposing that the reference points and the individual forces are set in the method similar to the first embodiment between the first reference point t₀ and the reference point of the time point t₃. Therefore, since step S51 to step S55 in FIG. 13 are similar to step S1 to step S5 in FIG. 6, description thereof is omitted.

As shown in (A) and (F) of FIG. 16, supposing that the guide wire 2 is pulled back at the time point t_{4′}. Every time the insertion length increases or decreases by the predetermined length, the reference point setting unit 16 compares the displacement of the force detected by the force detector 13 with the displacement of the force at the immediately preceding reference point in the method similar to the first embodiment. When the reference point setting unit 16 decides that the change occurs by the predetermined first threshold value or more, the reference point setting unit 16 sets the reference point t_{4′} (step S56).

When the reference point setting unit 16 decides at step S56 as being the reference point, the insertion length detector 15 decides whether the insertion length increases or decreases by the predetermined length (step S59).

When the insertion length detector 15 decides at step S59 that the insertion length increases, the decision result is output from the insertion length detector 15 to the database input/output unit 14 at step S60, and “1” is set in the column of the reference point in the measurement information database 9. Thereafter, the force measuring process goes to step S62.

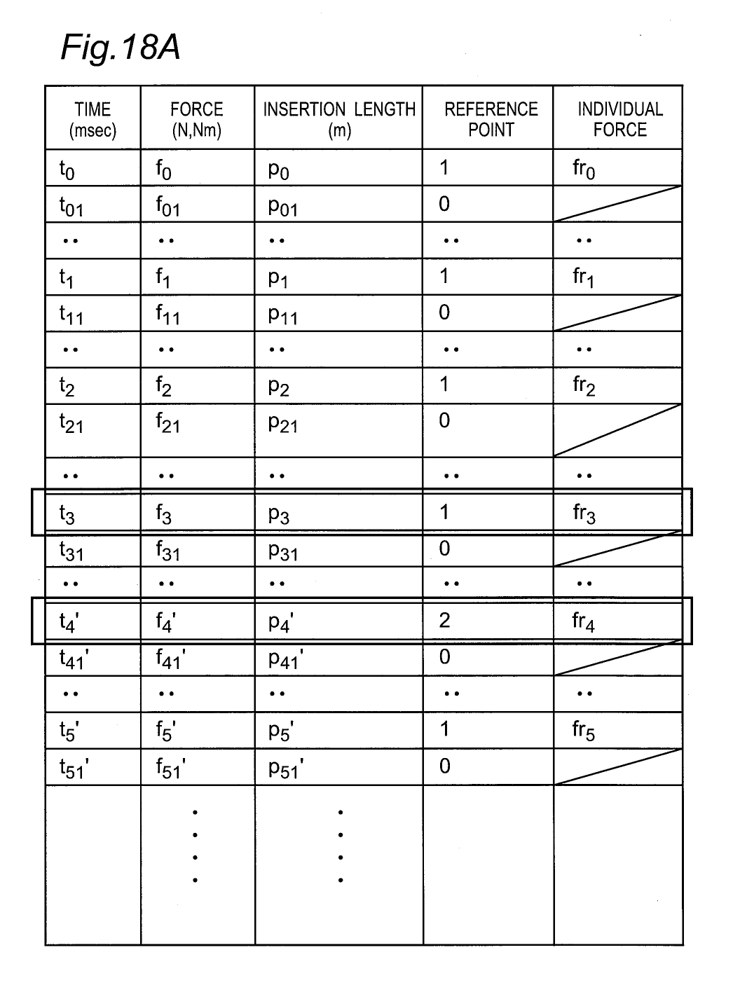

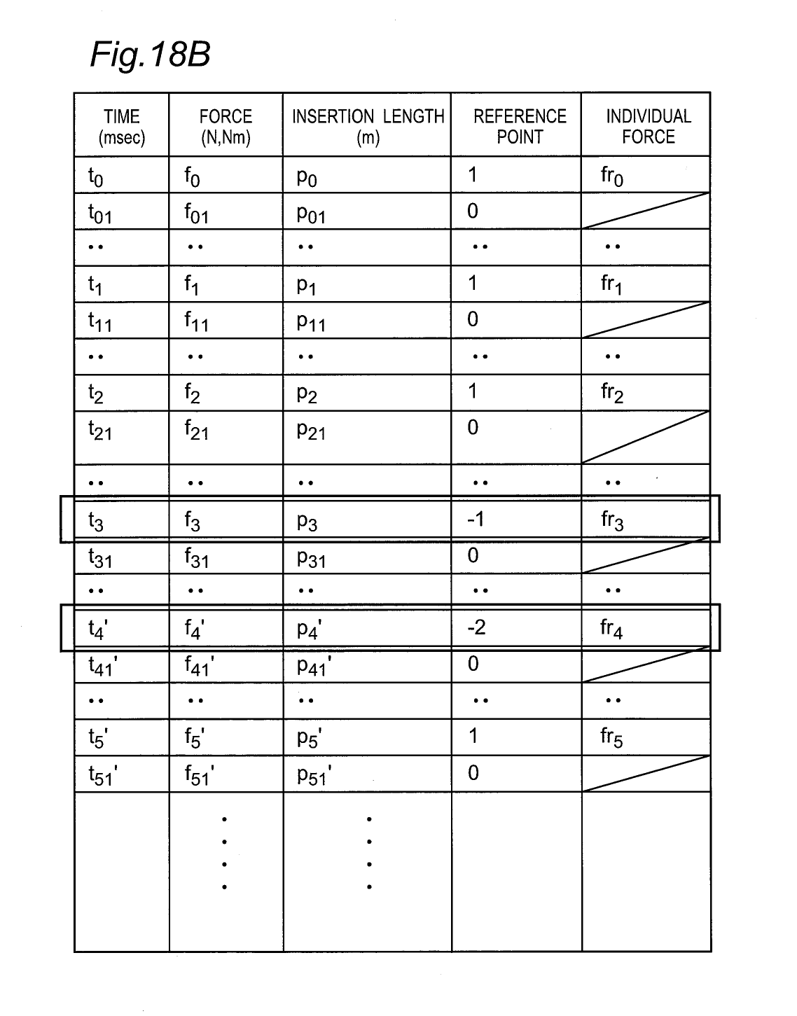

On the other hand, when the insertion length detector 15 decides at step S59 that the insertion length decreases, the decision result is output from the insertion length detector 15 to the database input/output unit 14 at step S61, and “2” is set in the column of the reference point in the measurement information database 9. Since the insertion length decreases at a time point t_{4′}, as shown in FIG. 18A, “2” is set in the column of the reference point for the time point t_{4′}. Thereafter, the force measuring process goes to step S62.

Next, the reference point correcting unit 17 decides at step S62 whether the reference point set at step S56 is a reference point after the next to the immediately preceding reference point with “2”. That is to say, the reference point correcting unit 17 checks whether the reference point is next to the time point when the pulling-back of the guide wire 2 is ended and the insertion is restarted.

When the reference point correcting unit 17 decides at step S62 that the reference point set at step S56 is the reference point after the next to the immediately preceding reference point with “2”, the force measuring process goes to step S63.

When the reference point correcting unit 17 decides at step S62 that the reference point set at S56 is not the reference point after the next to the immediately preceding reference point with “2”, the force measuring process goes to step S64. Since the time point t_{4′} set as the reference point before is not the reference point after the next to the immediately preceding reference point with “2”, the force measuring process goes to step S64. An example where the process goes to step S63 is described at time of calculating a time point t_{6′}, described later.

The individual force calculating unit 11 also counts the reference point with “2” at step S64 as the number of the reference points for the individual forces similarly to the reference point with “1”, so that the individual force calculating unit 11 calculates the individual forces. The result calculated by the individual force calculating unit 11 is stored in the measurement information database 9 from the individual force calculating unit 11 via the database input/output unit 14 (shown in FIG. 18A).

Further, a time point t_{5′} when the insertion is restarted is also calculated by the individual force calculating unit 11 using the similar method. That is to say, since the insertion length detector 15 decides at step S59 that the insertion length increases at the reference point t_{5′}, the reference point setting unit 16 sets “1” in the column of the reference point in the measurement information database 9 at step S60. Next, the individual force calculating unit 11 calculates an individual force at the reference point t_{5′} at step S64. A value Δf_{5′}, which is obtained by subtracting the force f_{4′} at the immediately preceding reference point t_{4′} from the force f_{5′} at the time point t_{5′}, is divided by the number of the reference points that have been set (in this example, the reference points are t₁, t₂, t₃, t_{4′}, and t_{5′} excluding the time point t₀, and thus the number of the reference points is “5”). The individual force calculating unit 11 calculates the obtained value f_{r5′}=Δf_{5′}/5 as the individual force at reference point t_{5′}. The individual force calculating unit 11 calculates individual forces at another reference points in a manner that an individual force f_{r5′} is added to the individual forces. The individual forces calculated by the individual force calculating unit 11 are output from the individual force calculating unit 11 to the database input/output unit 14, and are stored in the measurement information database 9 (in this example, they are stored in the measurement information database 9 in FIG. 18A). Thereafter, step S65 and step S66 are similar to step S11 and step S12 in FIG. 6.

<<Restart of Insertion at Time Point t_{5′}>>

Next, an operation for calculating a next reference point t_{6′} after the insertion at time point t_{5′} is restarted is described below.

The next reference point t_{6′} is calculated by the method similar to the first embodiment.

That is to say, since the insertion length detector 15 decides at step S59 that the insertion length increases, the reference point setting unit 16 sets “1” for the reference point of the time point t_{6′} at step S60.

Next, the reference point correcting unit 17 decides at step S62 whether the reference point set at step S56 is a reference point after the next to the immediately preceding reference point with “2”. Since the reference point with “2” just before the time point t_{6′} is the time point t_{4′}, the reference point correcting unit 17 decides that the reference point set at step S56 is a reference point after the next of the reference point t_{6′}, and the force measuring process goes to step S63.

At step S63, the reference point correcting unit 17 corrects the calculated reference points. The reference point correcting unit 17 searches for the reference point at which the insertion length becomes equal to or more than that at the time point t_{5′} at which the pulling-back is ended, sequentially starting from the time point t₀. In this example, the time point t₂ is found according to A17 of FIG. 17. The reference point correcting unit 17 deletes reference points until the reference point with “2” from the reference point after the obtained time point t₂. Concretely, the reference point correcting unit 17 updates information contents stored in the measurement information database 9 via the database input/output unit 14 so that the column of the reference point for the time point t₃ is corrected from “1” to “−1”, and the column of the reference point for the time point t₄ is corrected from “2” to “−2”. The corrected information contents in the measurement information database 9 are shown in FIG. 18B. After the correction in the reference point correcting unit 17, the individual force calculating unit 11 calculates the individual forces at step S64 based on the corrected reference points. The individual force calculating unit 11 calculates the individual forces using the reference points corrected by the reference point correcting unit 17 here, but reference points with minus signs such as the reference points with “−1” and “−2” are treated similarly to the reference point with “0”, and are calculated by the individual force calculating unit 11. That is to say, in the individual force calculating unit 11, the reference points before the pulling-back are used as the reference points until the time point t₂, and the reference point of the time point t_{5′} at which the insertion is restarted is used after the time point t₂, but the reference points between the time point t₂ and the time point t_{5′} are not used.

<<Effects of the Second Embodiment>>

Not only when the guide wire 2 is pushed into the blood vessel 3 but also when it is stopped and pulled out, the loads in these cases can be decided individually by the force deciding unit 12.

### Third Embodiment

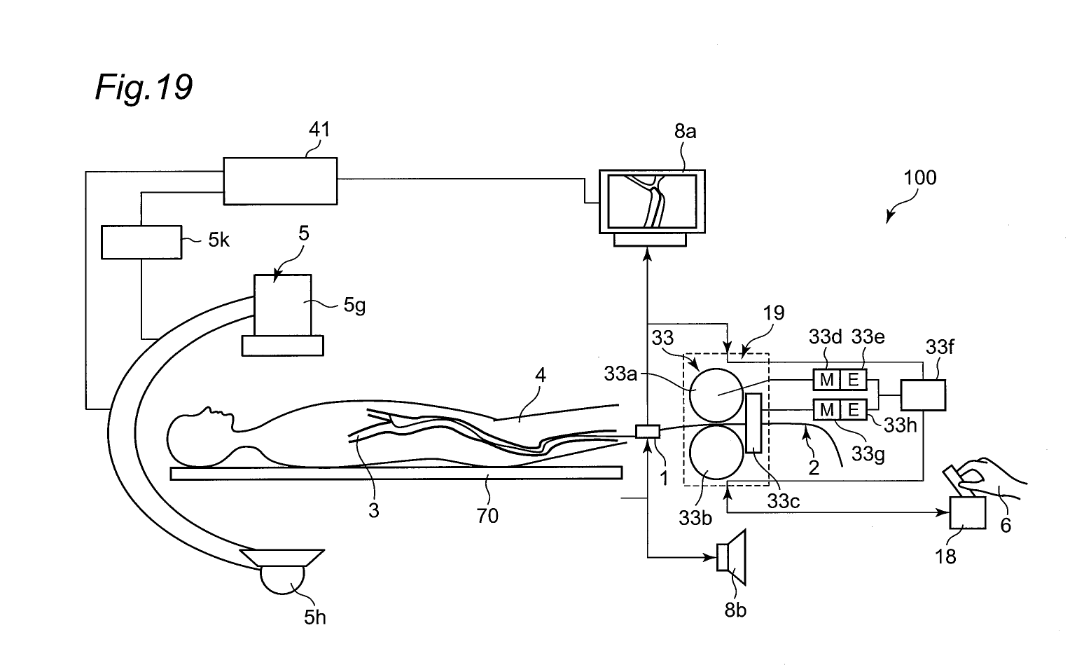

As to a force measurement apparatus 1C according to the third embodiment, as shown in FIG. 19, a case where a guide wire 2 is inserted into a blood vessel 3 by using a master slave apparatus 100 is described as an example.

A summary of the master slave apparatus 100 according to the third embodiment of the present invention is described first.

FIG. 19 illustrates a state of catheterization study or treatment with which a slave robot 19 inserts the guide wire 2 as one example of an insertion member into an affected area of a blood vessel 3 of a human body 4 such as a brain or a heart from the outside of the human body according to instructions from a hand of an operator 6 to a master robot 18.

While the operator 6 is manipulating the master robot 18 and allows the slave robot 19 to insert the guide wire 2, the blood vessel 3 or the guide wire 2 are imaged on the outside of the human body 4 by an X-ray imaging device 5, and the imaged image is displayed on a monitor 8a.

Further, the force measurement apparatus 1C measures a contact force at time the guide wire 2 contacts with the blood vessel 3 or a frictional force at time the guide wire 2 contacts with each of a meandering portion of the blood vessel 3 when the operator 6 operates the master robot 18 to insert the guide wire 2. When a load is applied to the blood vessel 3, the monitor 8a or a speaker 8b notifies the operator 6 of a warning. Further, when the individual forces measured by the force measurement apparatus 1C are fed back from the slave robot 19 to the master robot 18, the operator 6 has a feeling of force sensitive such that the operator 6 directly holds to manipulate the guide wire 2 with a hand. Further, the operator 6 can instruct the insertion of a catheter while checking an X-ray image displayed on the monitor 8a and the warning from the force measurement apparatus 1C. Further, the instructions for starting and ending the detection in the force measurement apparatus 10 are issued by manipulating the master robot 18 in cooperation with the start and end of the insertion work by the slave robot 19.

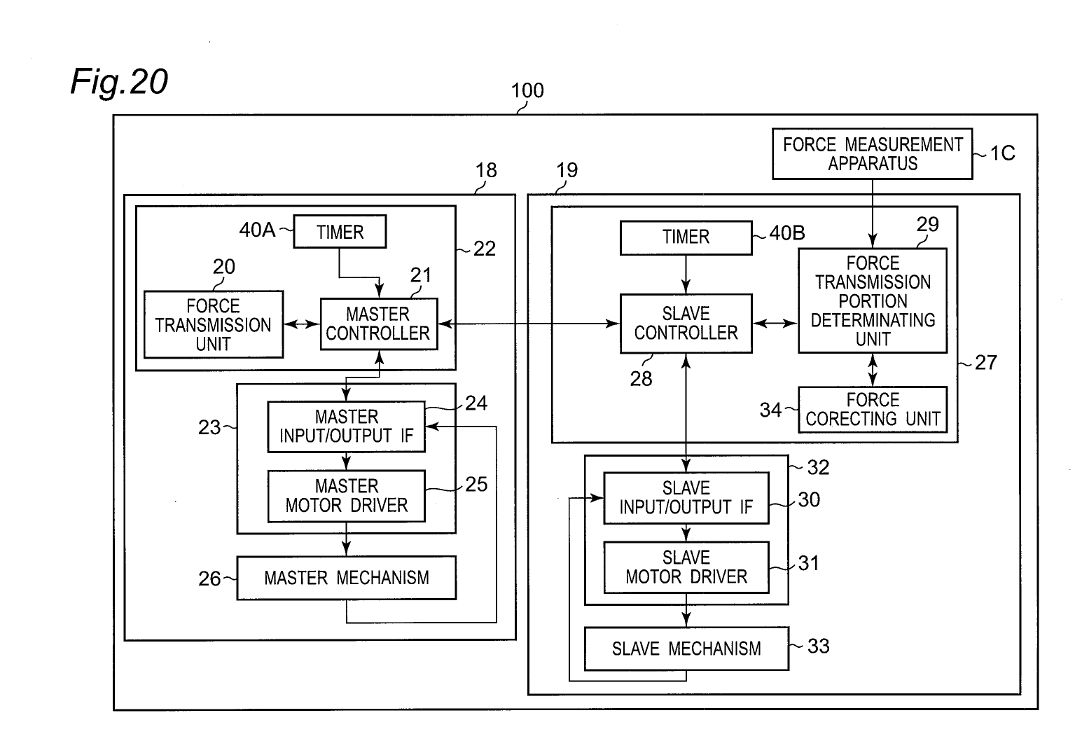

The force measurement apparatus 10, the master robot 18, and the slave robot 19 according to the third embodiment are described in detail below. FIG. 20 is a constitutional view illustrating the force measurement apparatus 1C, the master robot 18, and the slave robot 19.

<<Master Slave Apparatus 100, Master Robot 18, and Slave Robot 19>>

The master slave apparatus 100 is a whole apparatus which includes the force measurement apparatus 1C, the master robot 18, and the slave robot 19, and this apparatus can be manipulated remotely by a person when works are conducted. The master robot 18 is a robot system which is directly touched and manipulated by a person, and is composed of a master mechanism 26, a master control device 22, and a master peripheral device 23. The slave robot 19 is separated from the master robot 18, and is a robot system to be used for actually conducting works, and is composed of a slave mechanism 33, a slave control device 27, and a slave peripheral device 32.

<<Master Mechanism 26 and Slave Mechanism 33>>

The master mechanism 26 is a robot that is directly touched and manipulated by a person (the operator), and obtains position information at every sample time at time of manipulation of the person through a sensor (not shown) so as to output the position information to a master input/output IF 24.

The slave mechanism 33 is a robot that conducts a work for feeding the guide wire 2 as one example of the insertion member to the blood vessel 3, and moves according to the position information obtained by the master mechanism 26.

The slave mechanism 33 is a roller delivery device that moves to two axial directions, such as an insertion direction and a rotating direction around the insertion direction as a center axis. The slave mechanism 33 grips a flexible insertion member such as the guide wire 2 with an upper roller (first roller) 33a and a lower roller (second roller) 33b, and controls the motions of the rollers 33a and 33b so as to deliver the guide wire 2. The roller to be controlled here can be any one of the upper roller 33a and the lower roller 33b. The roller to be controlled is disposed with a motor 33d and an encoder 33e similarly to a joint portion of a robot arm, and is controlled by a motor driver 33f similarly to the robot arm. The upper roller 33a and the lower roller 33b are supported onto a pedestal, not shown, so as to be rotatable. Further, a third roller 33c is provided, and the third roller 33c can control to rotate the delivery unit composed of the upper roller 33a and the lower roller 33b around the insertion direction as the center axis. A bracket, not shown, is fixed to the third roller 33c, and the upper roller 33a and the lower roller 33b are supported to the bracket so as to be rotatably. The third roller 33c is provided with a motor 33g and an encoder 33h similarly to the joint portion of the robot arm, and is controlled by the motor driver 33f similarly to the robot arm. The third roller 33c is supported to the pedestal, not shown, so as to be rotatable. As a result, the motion of the guide wire 2 can be controlled in the insertion direction and also in the rotating direction around the insertion direction as the center axis.

<<Timers 40A and 40B>>

A master controller 21 or a slave controller 28 is started by timers 40A and 40B after a certain time elapses (for example, every 1 msec).

<<Master Peripheral Device 23 and Slave Peripheral Device 32>>

The master peripheral device 23 is composed of the master input/output IF 24 and the master motor driver 25, and transmits information between the master mechanism 26 and the master control device 22.

Similarly, the slave peripheral device 32 is composed of a slave input/output IF 30 and a slave motor driver 31, and transmits information between the slave mechanism 33 and the slave control device 27.

The master input/output IF 24 outputs the position information from the master mechanism 26 to the master controller 21. Further, the position information from the master controller 21 is output to the master motor driver 25 at every certain constant time (for example, 1 msec) using the timer 40A. The master motor driver 25 drives a motor of the master mechanism 26 according to the position information from the master input/output IF 24.

The slave input/output IF 30 outputs the position information from the slave controller 28 to the slave motor driver 31. Further, the position information from the slave mechanism 33 is output to the slave controller 28 at every certain constant time (for example, 1 msec) using the timer 40B. The slave motor driver 31 drives the motor of the slave mechanism 33 according to the position information from the slave input/output IF 30.

<<Master Control Device 22, Slave Control Device 27>>

The master control device 22 is composed of the timer 40A, a force transmission unit 20, and the master controller 21. The master control device 22 has two roles. One of the roles is for outputting the position information about the motion of the master mechanism 26 to the slave control device 27 at every certain constant time (for example, 1 msec) using the timer 40A. The other role is for transmitting the force information input from the slave control device 27 to the person (the operator). The master controller 21 outputs the position information about the master mechanism 26 from the master input/output IF 24 to the slave controller 28 at every certain constant time (for example, 1 msec) using the timer 40A. Further, the force information from the slave controller 28 is output to the force transmission unit 20. The force transmission unit 20 transmits the force information from the slave controller 28 to the hand of the operator 6. The direction where the force is generated includes two directions, that is, the insertion direction of the master mechanism 26 and the rotating direction around the insertion direction.

The slave control device 27 is composed of the timer 40B, the slave controller 28, a force transmission portion determining unit 29, and a force correcting unit 34. The slave control device 27 has two roles. One of the roles is for making the slave controller 28 tracking-control of the slave mechanism 33 according to the position information from the master control device 22. The other role is for making the force transmission portion determining unit 29 determine a force to be transmitted to the master control device 22 based on the force information obtained by the force measurement apparatus 1C, and making the force correcting unit 34 correct the determined force so as to output the corrected force as the force information to the master control device 22. The force measurement apparatus 1C is arranged near the place where the slave robot 19 is arranged outside the human body (patient) 4 as shown in FIG. 19.

<<Force Measurement Apparatus 1C>>

The force measurement apparatus 1C has the function equivalent to that in the first embodiment or the second embodiment. For example, the force measurement apparatus 1C can be composed of the force measurement apparatus 1, the force measurement apparatus 1B, or a force measurement apparatus according to an embodiment described later. An output value from a force detector 13, all individual forces calculated by an individual force calculating unit 11, and a decided result in a force deciding unit 12 are output from the force measurement apparatus 1C to the force transmission portion determining unit 29 of the slave control device 27, described later.

<<Force Transmission Portion DE Terminating Unit 29>>

The force transmission portion determining unit 29 determines, among the individual forces calculated by the individual force calculating unit 11 of the force measurement apparatus 1C and forces in the force detector 13, a force to be transmitted to the master control device 22 based on a determination flag held inside. When the force is transmitted to the force detector 13, “0” is set in the determination flag, and when a value, which is obtained by subtracting a force at a reference point determined the most recently among the individual forces in the force measurement apparatus 1C from the force of the force detector 13 at present (measurement time point) is transmitted, “1” is set.

<<Force Correcting Unit 34>>

The force correcting unit 34 performs smoothing so that the force does not suddenly changes at a time point when the determination flag is switched by the force transmission portion determining unit 29, namely, the force smoothly switches from the force before switching into a force after switching.

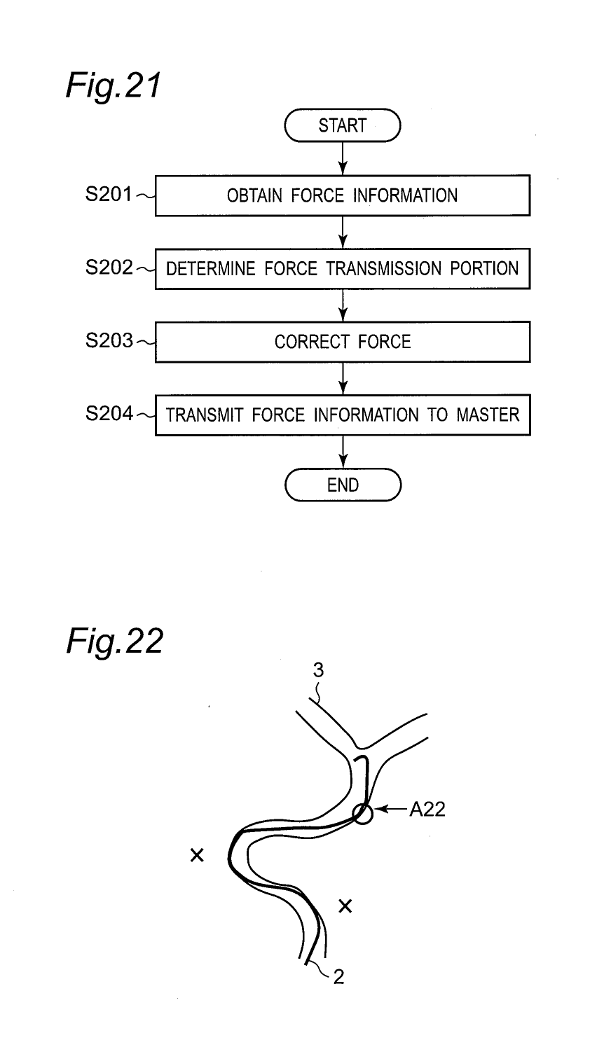

A manipulating procedure in the master slave apparatus 100 according to the third embodiment is described with reference to a flowchart of FIG. 21.

A procedure at time when the operator 6 directly touches the master mechanism 26 to manipulate the slave mechanism 33 so as to deliver the guide wire 2, the guide wire 2 contacts with the blood vessel 3 is described with reference to FIG. 21.

When the guide wire 2 contacts with the blood vessel 3, the force information is detected by the force detector 13 of the force measurement apparatus 1C, and is output from the force detector 13 to the force transmission portion determining unit 29 at step S201. The force transmission portion determining unit 29 determines that the force in the force detector 13 is transmitted to the slave controller 28 when the determination flag held inside indicates “0”. When the determination flag held inside indicates “1”, the force transmission portion determining unit 29 makes a determinations so that the value, which is obtained by subtracting the force at reference point determined the most recently among the individual forces in the force measurement apparatus 1C from the force in the force detector 13 at the present (measurement time point), is transmitted from the force transmission portion determining unit 29 to the slave controller 28. When the determination flag indicates “0”, the force in the force detector 13 (a summed-up value of the contact forces at all the portions) is transmitted from the force transmission portion determining unit 29 to the slave controller 28. For this reason, a force equivalent to the force at time the operator 6 conventionally directly grips the guide wire 2 is transmitted from the force transmission portion determining unit 29 to the slave controller 28. When the determination flag indicates “1”, only the most recent contact force is transmitted from the force transmission portion determining unit 29 to the slave controller 28. For this reason, the force of only a portion having influence at the present (measurement time point) can be transmitted from the force transmission portion determining unit 29 to the slave controller 28 regardless of the meandering state or the contact state until this time point. For example, as shown in FIG. 22, when the direction towards the branch portions is desired to be changed using the force at the distal end of the guide wire as a fulcrum as shown in FIG. 22, the operator can perform the manipulation while feeling the force of only a portion A22 in FIG. 22.

At step S203, the force correcting unit 34 executes the smoothing so that the force does not suddenly changes at the time point when the determination flag is switched by the force transmission portion determining unit 29, namely, the force smoothly switches from the force before switching into the force after switching.

At step S204, the force information output to the slave controller 28 is sent to the master controller 21 via wireless or wired communication unit so as to be transmitted to the force transmission unit 20. The force information input into the force transmission unit 20 is transmitted to the hand of the operator 6.

<<Effect of the Third Embodiment>>

When the slave robot 19 inserts the guide wire 2 as one example of the insertion member into an affected area of the blood vessel 3 of the human body 4 such as a brain or a heart, from the outside of the human body according to the instructions from the operator 6 to the master robot 18, the force to be transmitted can be switched into only the force equivalent to the force at the time the operator 6 conventionally directly grips the guide wire 2 or only the most recent contact force. In the former case, the operator 6 can feel the force at the time of conventionally directly gripping the guide wire 2. In the latter case, regardless of the meandering state or the contact state, only the force on only a portion having influence at the present (measurement time point) can be transmitted.

### Fourth Embodiment

Similarly to the third embodiment, a case where a force measurement apparatus 1D according to the fourth embodiment inserts a guide wire 2 into a blood vessel 3 using a master slave apparatus 100D is described as an example as shown in FIG. 19. Description about the portions in the fourth embodiment that are common with the first, second and third embodiments is omitted, and only different portions are described in detail below. Similarly to the force measurement apparatus 1C, the force measurement apparatus 1D is composed of any one of the force measurement apparatus 1, the force measurement apparatus 1B, and a force measurement apparatus according an embodiment described later.

A summary of the master slave apparatus 100D according to the fourth embodiment is described first with reference to FIG. 19.

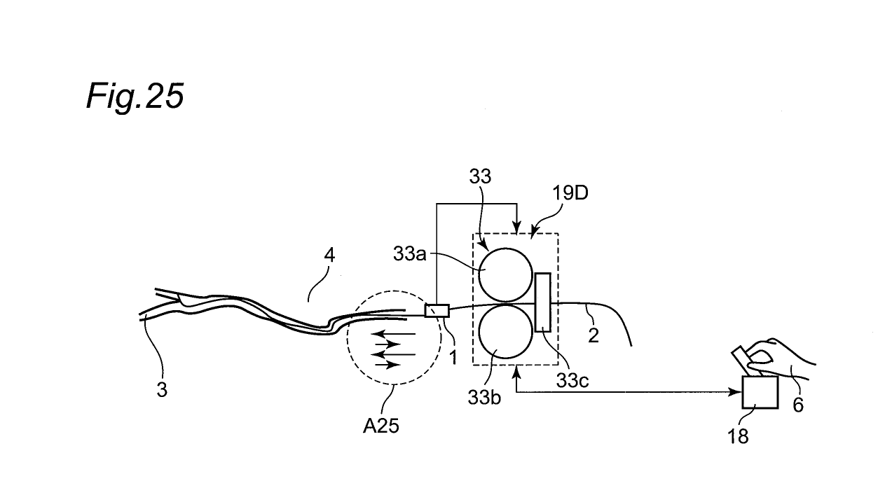

While the operator 6 is manipulating a master robot 18 so as to insert the guide wire 2, the force measurement apparatus 1D measures a contact force at time the guide wire 2 contacts with the blood vessel 3 or a frictional force at time the guide wire 2 contacts with each of the meandering portions etc. of the blood vessel 3 when the operator 6 manipulates the master robot 18 to insert the guide wire 2. When a load is applied to the blood vessel 3, a monitor 8a or a speaker 8b notifies the operator 6 of a warning, and in addition thereto, a slave robot 19D stops the control of the slave. Further, when the guide wire 2 is clogged in the blood vessel 3 and the guide wire 2 cannot further advance in the blood vessel 3, the slave robot 19D makes a vibration motion, described later, so as to remove the clogging of the guide wire 2 from the blood vessel 3 and enables the guide wire 2 to advance. The vibration control is motion for vibrating the guide wire 2 with respect to the blood vessel 3 as shown by A25 in FIG. 25. In this control, after making the guide wire 2 slightly advance in the blood vessel 3, the slave robot 19D makes the guide wire 2 slightly retreat in the blood vessel 3, and the advance and retreat are repeated. Further, the operator 6 can instruct the catheter insertion while checking an X-ray image displayed on the monitor 8a and a warning etc. from the force measurement apparatus 1D similarly to the third embodiment. Further, the instructions for starting and ending the detection in the force measurement apparatus 1D is issued in cooperation with the start and the end of the insertion work to be done by the slave robot 19D through the manipulation of the master robot 18.

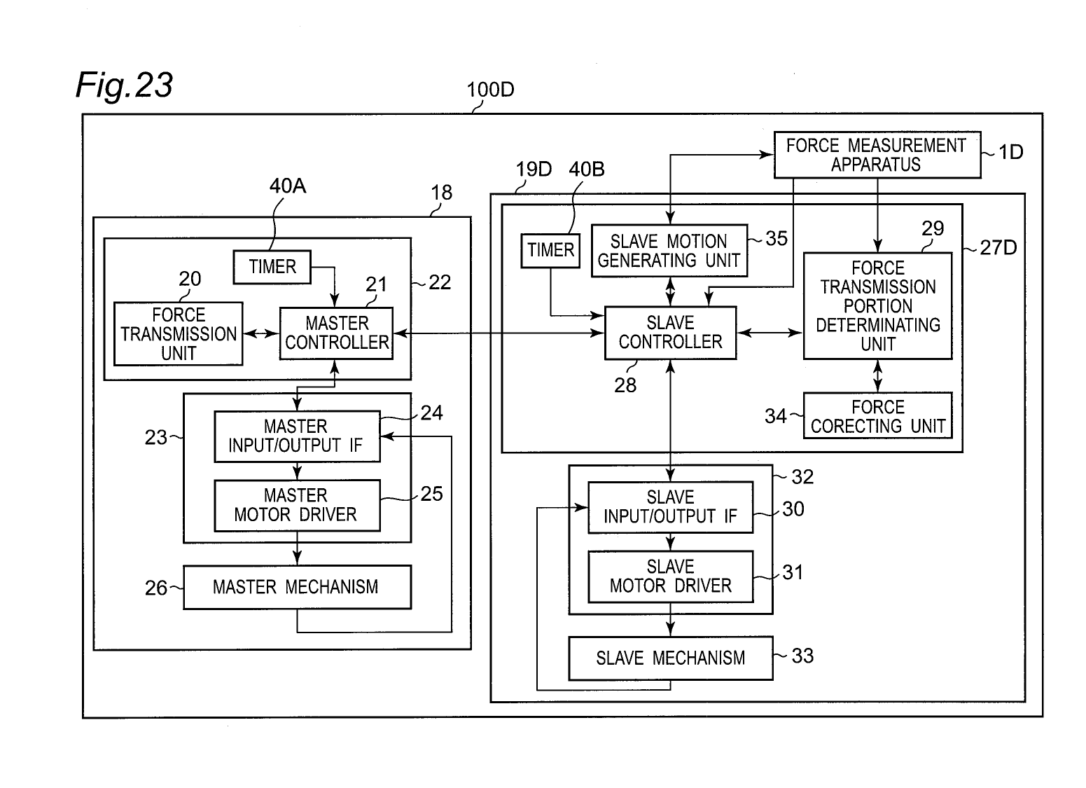

Next, the force measurement apparatus 1D, the master robot 18, and the slave robot 19D according to the fourth embodiment are described in detail below. FIG. 23 is a constitutional view illustrating the force measurement apparatus 1D, the master robot 18, and the slave robot 19D. Description about the portions in the fourth embodiment that are common with the third embodiment is omitted, and only different portions are described in detail below.

<<Slave Mechanism 33>>

The slave mechanism 33 is a robot that does a work for delivering the guide wire 2 as one example of the insertion member to the blood vessel 3. The slave mechanism 33 performs tracking control based on the position information obtained by a master mechanism 26, and makes a motion generated by a slave motion generating unit 35, described later. The slave mechanism 33 of the slave robot 19D in FIG. 25 has the constitution similar to the slave mechanism 33 in FIG. 19, and detailed illustration is omitted.

<<Slave Control Device 27D>>

The slave control device 27D has three roles. The first role is to make the slave mechanism 33 follow the position information from the master control device 22. The second role is to determine a force to be transmitted to the master control device 22 by the force transmission portion determining unit 29 based on the force information obtained by the force measurement apparatus 1D, correct the determined force through a force correcting unit 34, and output the corrected force as the force information to the master control device 22. The third role is to make control based on the motions generated by the slave motion generating unit 35. The force measurement apparatus 1D is arranged near the place where the slave robot 19D is arranged outside a human body 4 as shown in FIG. 19.

<<Slave Motion Generating Unit 35>>

The slave motion generating unit 35 generates a motion for stopping a slave motion and a motion for vibrating the slave based on the force information obtained by the force measurement apparatus 1D or the load decided result. The vibration control is a motion for inserting and returning the guide wire 2 with respect to the blood vessel 3 repeatedly little by little as shown by A25 in FIG. 25 through the slave robot 19D. Concretely, the guide wire 2 is advance with respect to the blood vessel 3 by a constant first insertion length (for example, 3.6 mm) for the predetermined first time (for example, 60 msec), and the guide wire 2 is retreated with respect to the blood vessel 3 by a constant second insertion length (for example, 0.3 mm) for the predetermined second time (for example, 10 msec) in a repeated manner.

When the force deciding unit 12 of the force measurement apparatus 1D decides that a load is applied, a command for stopping the slave motion is given to the slave controller 28. Further, a parameter of the vibration control is changed according to the strength of the force obtained by the force measurement apparatus 1D. For example, when the obtained force is strong, a vibration cycle at the time of the vibration control is made to be longer (for example, the predetermined first time is 30 msec), or an amplitude of vibration is increased (for example, the first insertion length is 6 mm). When the obtained force is weak, the vibration cycle at the time of the vibration control (for example, the predetermined first time is 80 msec) is reduced, and the amplitude of vibration is reduced (for example, the first insertion length is 2 mm).

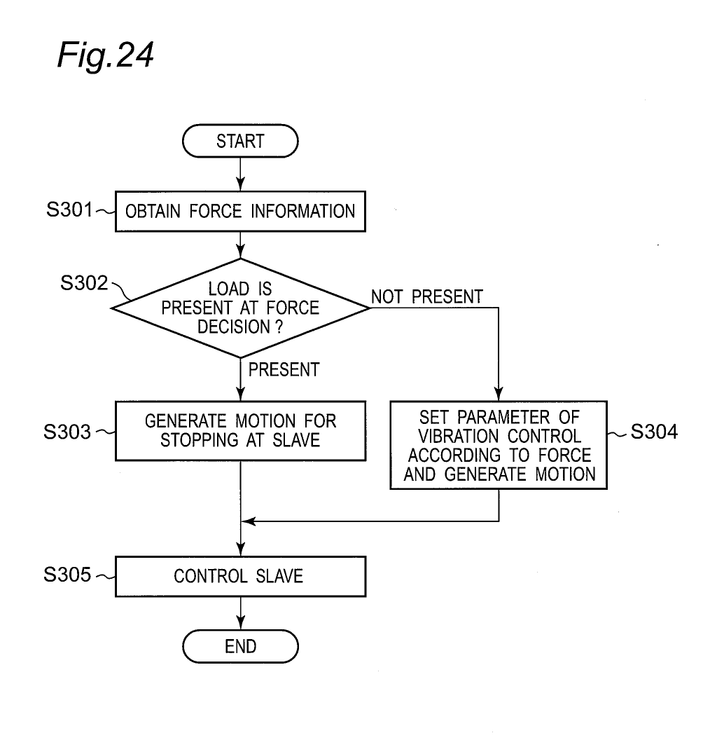

A manipulating procedure in the master slave apparatus 100D according to the fourth embodiment is described with reference to a flowchart of FIG. 24.

A procedure for controlling the slave mechanism 33 at time the guide wire 2 contacts with the blood vessel 3 when the operator 6 directly touches the master mechanism 26 and manipulates the slave mechanism 33 that delivers the guide wire 2 is described with reference to FIG. 24.

At step S301, when the guide wire 2 contacts with the blood vessel 3, the force information is detected by a force detector 13 of the force measurement apparatus 1D and is output from the force detector 13 to the slave motion generating unit 35.

When the force deciding unit 12 of the force measurement apparatus 1D decides at step S302 that a load is present, the slave motion generating unit 35 issues a command for stopping the slave motion from the force deciding unit 12 to the slave controller 28 (step S303). Thereafter, the sequence goes to step S305.

When the force deciding unit 12 of the force measurement apparatus 1D decides at step S302 that a load is not present, the slave motion generating unit 35 changes the parameter of the vibration control according to the strength of the force obtained by the force measurement apparatus 1D. When the obtained force is strong, for example, the slave motion generating unit 35 lengthens the vibration cycle at the time of the vibration control or increases the amplitude of vibration. When the obtained force is weak, the slave motion generating unit 35 shortens the amplitude cycle at the time of vibration control or decreases the amplitude of vibration (step S304). Thereafter, the sequence goes to step S305.

Next, at step S305, the slave mechanism 33 is controlled by the command from the slave motion generating unit 35.

<<Effect of the Fourth Embodiment>>

When a load is applied to the blood vessel 3, the monitor 8a or the speaker 8b gives a warning, and the slave control can be stopped by the slave robot 19D. Further, when the guide wire 2 is clogged in the blood vessel 3 and the guide wire 2 cannot further advance, the slave robot 19D makes the vibration motion so that the clogging of the guide wire 2 in the blood vessel 3 can be eliminated so that the guide wire 2 can advance.

### Fifth Embodiment

A summary of a force measurement apparatus 1E according to the fifth embodiment is described.

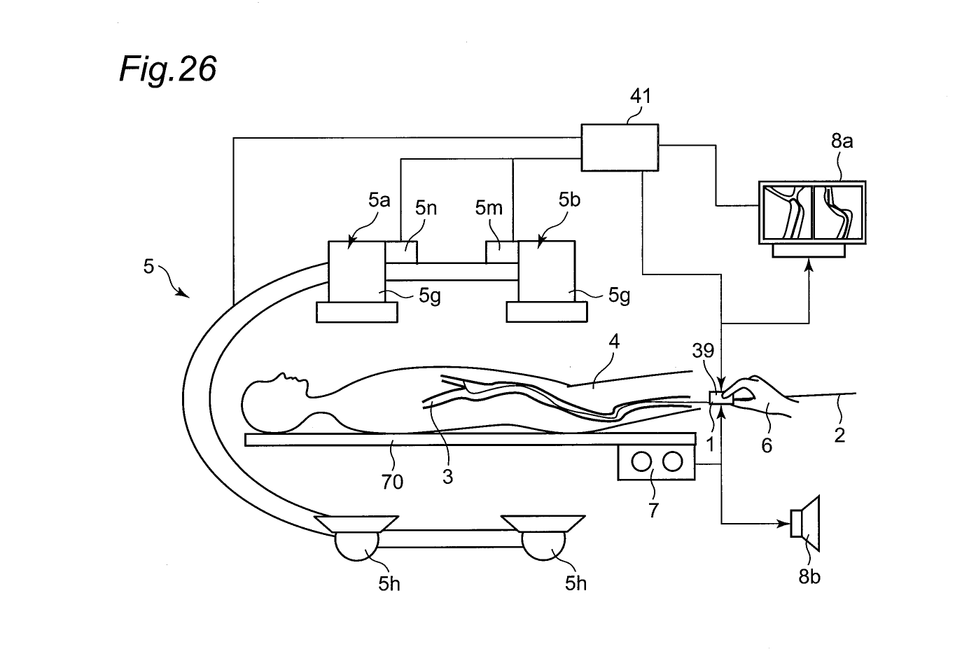

FIG. 26 illustrates a state of catheterization study or treatment with which an operator 6 inserts a guide wire 2 as one example of an insertion member into an affected area of a blood vessel 3 of a brain or a heart from the outside of a human body 4.

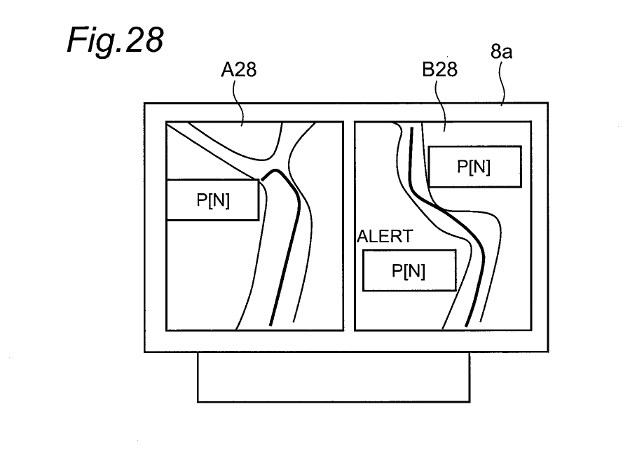

While the operator 6 is inserting the guide wire 2 into the blood vessel 3, a first X-ray imaging device 5a and a second X-ray imaging device 5b as one example of the imaging device image the blood vessel 3 or the guide wire 2 on the outside of the human body, and the imaged images are displayed on two screens of a monitor 8a via an X-ray imaging controller 41. One of the screens on the monitor 8a (see A28 of FIG. 28) displays the distal end of the guide wire 2 imaged by the first X-ray imaging device 5a. When individual forces are measured by the force measurement apparatus 1E and a load is applied to the blood vessel 3, the second X-ray imaging device 5b is moved to the portion to which the load is applied by a second X-ray imaging device transfer unit 5n, so that the image of the portion is displayed on the other one of the screens (see B28 in FIG. 28). Further, an image may be displayed so that the portion to which a load is applied can be recognized on the entire human body 4. Each of the first X-ray imaging device 5a and the second X-ray imaging device 5b have an X-ray generator 5g and an X-ray detector 5h corresponding to the X-ray generator 5g similarly to the X-ray imaging device 5 according to the first embodiment. Further, the speaker 8b gives a warning. The first X-ray imaging device 5a is transferred to a desired position by a first X-ray imaging device transfer unit 5m, and the second X-ray imaging device 5b is transferred to another desired position by the second X-ray imaging device transfer unit 5n under the control of the X-ray imaging controller 41.

The operator inserts the catheter while checking the X-ray images on the two screens on the monitor 8a and the warning from the force measurement apparatus 1E.

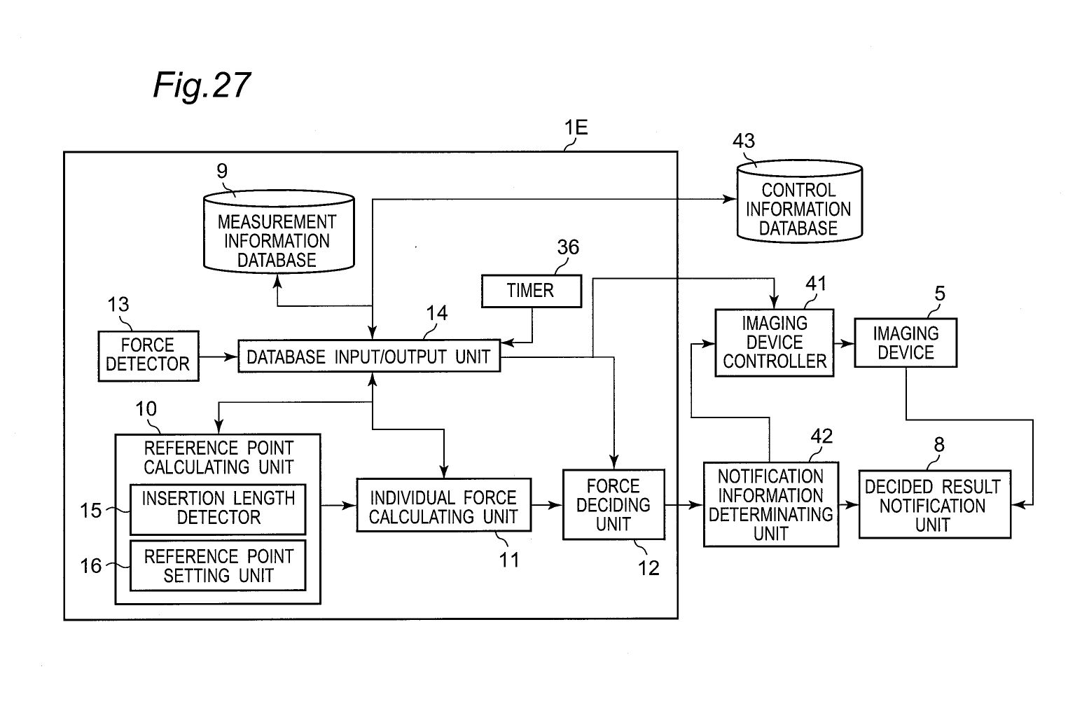

FIG. 27 is a constitutional view illustrating the force measurement apparatus 1E, the decided result notification unit 8, and the imaging device controller 41, a notification information determining unit 42, the imaging device 5, and a control information database 43 according to the fourth embodiment. Since the force measurement apparatus 1E excluding the force deciding unit 12 is similar to the force measurement apparatus 1 according to the first embodiment, description thereof is omitted.

<<Force Deciding Unit 12>>

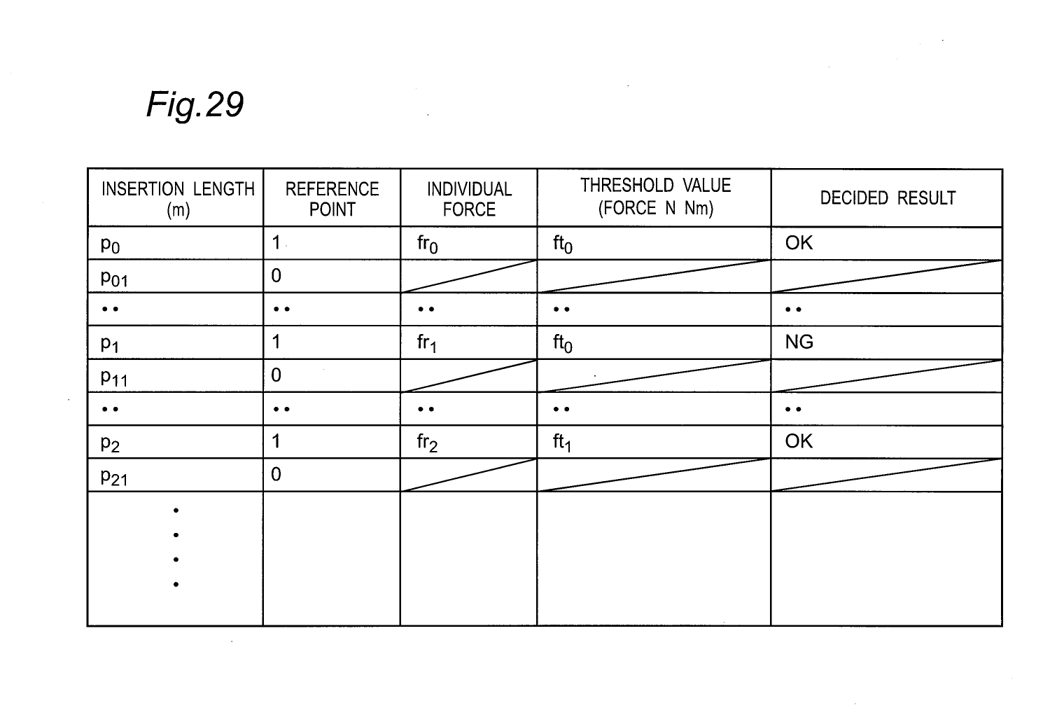

When a force calculated by an individual force calculating unit 11 is a predetermined second threshold value (for example, 0.5 N) or more, the force deciding unit 12 decides that a load is applied to the blood vessel 3. FIG. 29 illustrates one example of information about the decided result output from the force deciding unit 12. As shown in FIG. 29, the decided result as well as the force calculated by the individual force calculating unit 11, a predetermined threshold value used in the decision, an insertion length, and a reference point is output to the notification information determining unit 42.

<<Notification Information DE Terminating Unit 42>>

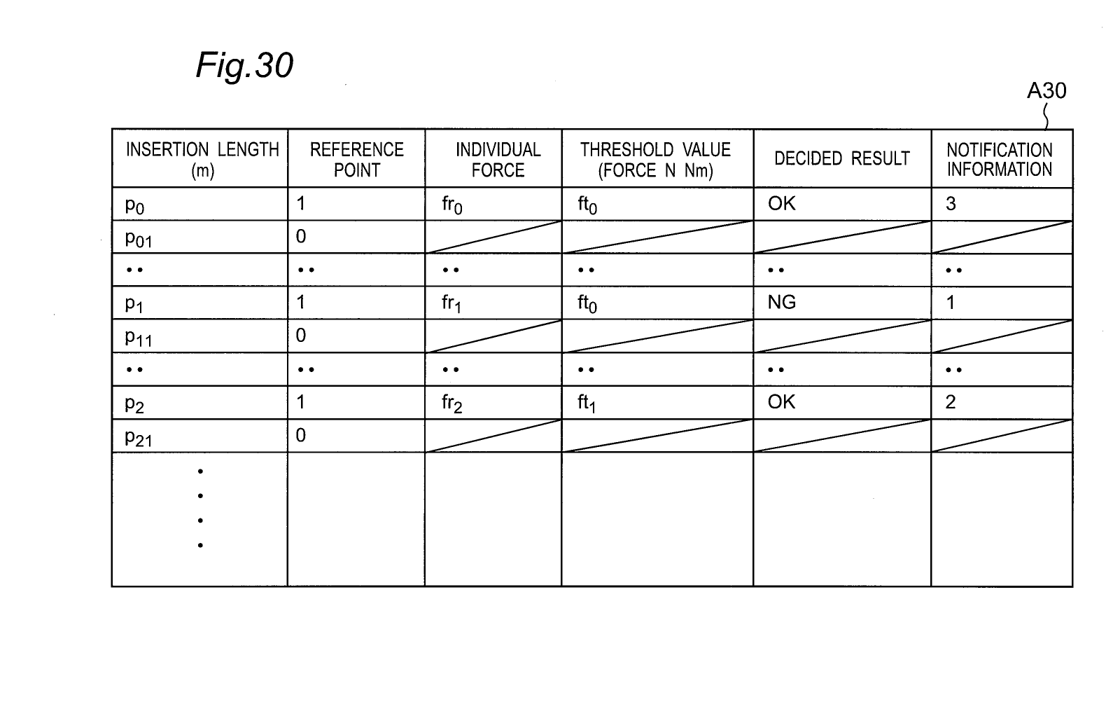

The notification information determining unit 42 determines notification information notified by the decided result notification unit 8, described later, based on the information about the decided result decided by the force deciding unit 12. FIG. 30 illustrates one example of the notification information (A30 of FIG. 30) determined by the notification information determining unit 42 in addition to the decided result output from the force deciding unit 12. The notification information determining unit 42 determines priorities of the information to be notified as “1”, “2”, . . . in decreasing order of priority, and the information about the decided result detected by the force deciding unit 12 and the notification information are output from the notification information determining unit 42 to the imaging device controller 41 and the decided result notification unit 8. As one example of the notification information, the priorities of portions which are decided that a load is present, namely, decided as “NG” by the force deciding unit 12 are determined in the order of a difference between the threshold value and the individual forces from largest to smallest. When no “NG” portion is present, the priorities of the portions are set in the order of the insertion length from longest to shortest. When one “NG” portion is present, the priority of the “NG” portion is determined as highest, and the priorities of the residual portions are determined in the order of the insertion length from longest to shortest.

<<Decided Result Notification Unit 8>>

The decided result notification unit 8 allows the notification information determined by the notification information determining unit 42 to be displayed on the monitor 8a in the decreasing order of priority. In this example, since the monitor 8a has the two screens as shown in FIG. 28, the first screen necessarily displays the distal end of the guide wire 2 (see A28 in FIG. 28), and only the second screen displays information with the highest priority (see B28 in FIG. 28). As the information to be displayed, a force P [N] calculated by the individual force calculating unit 11 is displayed, and when the force deciding unit 12 decides that a load is applied to the blood vessel 3, a recognizable warning such as “ALERT” is displayed. In this example, the first screen necessarily displays the distal end of the guide wire 2, but the information about the priorities such as “1” and “2” may be displayed on the respective screens. Further, in this example, the two screens are displayed, but three or more screens may be displayed. Further, the information about the insertion length may be displayed similarly to the forces. With the decided result notification unit 8 (for example, an image processing unit contained in the monitor 8a), the warning such as “ALERT”, and the load are displayed on the second screen so as to be overlapped with the image of the portion to which the load is applied.

Further, when the force deciding unit 12 decides that a load is applied to the blood vessel 3, the speaker 8b may generate a warning sound so as to give a warning to the operator.

<<Control Information Database 43>>

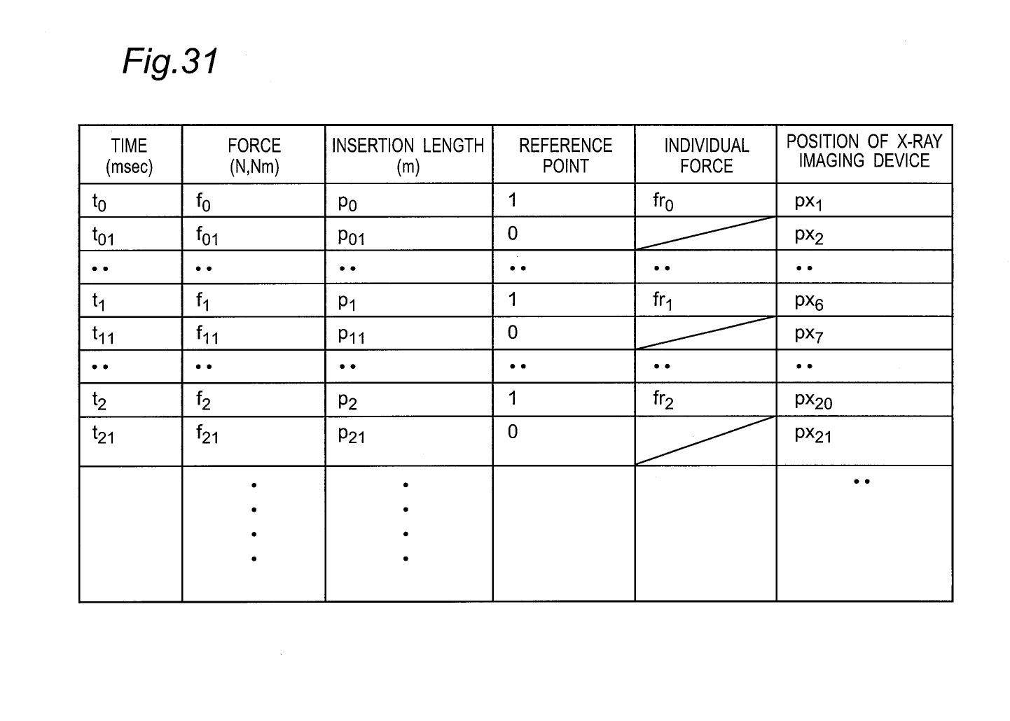

The control information database 43 records the position information about the first X-ray imaging device 5a and the second X-ray imaging device 5b in the imaging device controller 41 as well as information in a measurement information database 9 is recorded as shown in FIG. 31.

<<Imaging Device Controller 41>>

The imaging device controller 41 controls the positions of the first X-ray imaging device 5a and the second X-ray imaging device 5b, and obtains the positions of the first X-ray imaging device 5a and the second X-ray imaging device 5b at present (measurement time point) based on the notification information determined by the notification information determining unit 42.

Concretely, the first X-ray imaging device 5a is transferred by the operator 6 or a radioactive ray technician manually or by the first X-ray imaging device transfer unit 5m according to the insertion work for the guide wire 2 by the operator 6 so as to be capable of imaging the distal end of the guide wire 2. During the insertion work, individual forces are measured by the force measurement apparatus 1E similarly to the first embodiment. The imaging device controller 41 records the position of the transferred first X-ray imaging device 5a as well as the information in the measurement information database 9 into the control information database 43. Further, the first X-ray imaging device 5a is controlled so that the notification information determined by the notification information determining unit 42 is displayed. Since the first X-ray imaging device 5a is transferred by the operator 6 so as to image the distal end of the guide wire 2, the transfer is not controlled by the imaging device controller 41. The transfer of the second X-ray imaging device 5b is controlled by the imaging device controller 41 in order to image information with the highest priority.

For example, a case where the information about the insertion length “p1” with the highest priority is imaged by the second X-ray imaging device 5b and is displayed is described as an example with reference to FIG. 30. The imaging device controller 41 calculates the position of the second X-ray imaging device 5b at the insertion length “p1” based on the control information database 43. Concretely, in the control information database 43, the position of the second X-ray imaging device 5b at the insertion length “p1” is calculated by the imaging device controller 41. In an example of FIG. 31, the position of the second X-ray imaging device 5b at the insertion length “p1” is “px6”. Then, the imaging device controller 41 controls the transfer so that the position of the second X-ray imaging device 5b is “px6”.

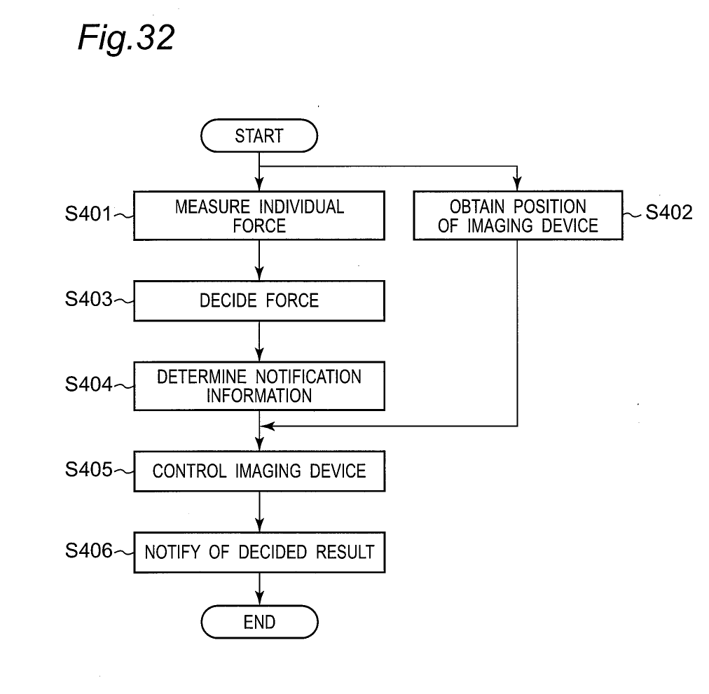

A procedure in the fifth embodiment is described with reference to a flowchart of FIG. 32.

At step S401, the individual force calculating unit 11 calculates individual loads at time the guide wire 2 contacts with the blood vessel 3. Thereafter, the force measuring process goes to step S403.

On the other hand, the imaging device controller 41 obtains the position at the time the operator 6 transfers the second X-ray imaging device 5b simultaneously with step S401, and records the position in the control information database 43 (step S402). Thereafter, the force measuring process goes to step S405.

At step S403, the force deciding unit 12 decides whether a load is applied based on the individual forces calculated by the individual force calculating unit 11.

At step S404, the notification information determining unit 42 determines information to be notified based on the decided result from the force deciding unit 12.

At step S405, the imaging device controller 41 controls the transfer of the second X-ray imaging device 5b to a portion to which the load is applied based on notification information determined by the notification information determining unit 42, and the second X-ray imaging device 5b images that portion. The imaging device controller 41 displays the information imaged by the second X-ray imaging device 5b, and also the notification information determined by the notification information determining unit 42 on the monitor 8a of the decided result notification unit 8.

<<Effect of the Fifth Embodiment>>

The X-ray image of the distal end of the guide wire 2 and the X-ray image of the portion to which the load is applied can be displayed simultaneously.

<<Modification Examples of the Respective Embodiments>>

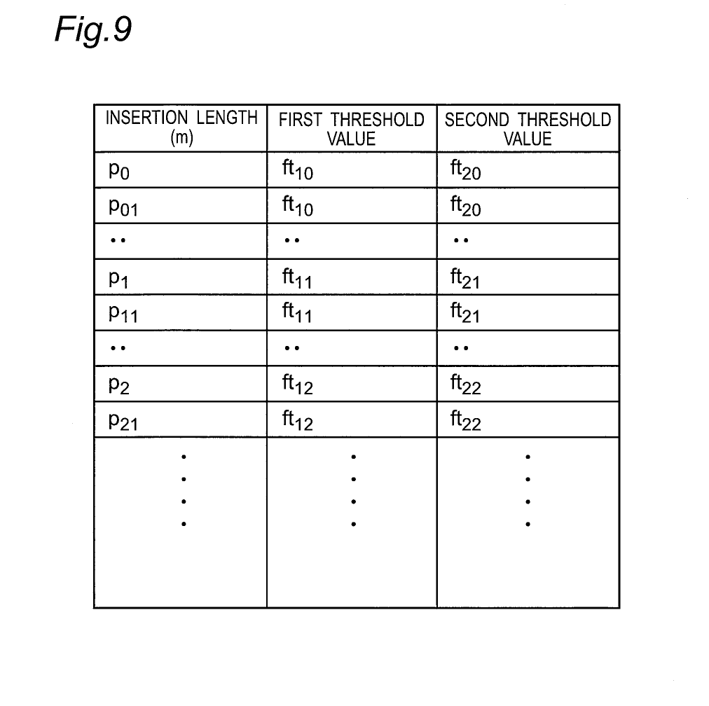

In the first embodiment, the reference point calculating unit 10 or the force deciding unit 12 provides the predetermined threshold value (the first threshold value or the second threshold value), but as shown in FIG. 9, the threshold value may be changed according to the insertion length. For example, when the guide wire 2 is inserted into the blood vessel 3 of a groin, the blood vessel 3 becomes thinner as the insertion of the guide wire 2 proceeds. For this reason, when the insertion of the guide wire 2 is started, the threshold value is set large, and the threshold value can be set small because the blood vessel 3 becomes thinner as the insertion of the guide wire 2 proceeds. Further, the threshold value may be individually corrected for each treatment method and each patient (the human body 4).

Further, in the first embodiment, the reference point calculating unit 10 calculates a time point when the displacement of a force is the predetermined threshold value or more at each predetermined insertion length, as the reference point. The individual force calculating unit 11 makes a calculation in a manner that information about a force at the immediately preceding reference point is subtracted from each force at each reference point, the obtained value is divided by the number of the reference points that have been set, and adds the obtained value to the individual forces at the respective reference points equally. In a method different from the above one, when a reference point is set at each predetermined time and a value obtained by the division by the number of the reference points that have been set is equally added to the individual forces at the reference points, the value may be added only to the forces at reference points that are the predetermined threshold value or more.

Further, the individual force calculating unit 11 subtracts the information about the force at the immediately preceding reference point from the information about a force detected by the force detector 13, divides the obtained value by the number of the reference points that have been set, and adds the obtained value to the individual forces at the respective reference points equally. However, the values to be added are not equally added but the values to be added may be individually changed according to the travel distance of the distal end of the guide wire 2. For example, when the distal end of the guide wire 2 transfers by the similar quantity to the insertion length, the individual forces at the reference points do not change. The value, which is obtained by subtracting the information about the force at the immediately preceding reference point from the information about the force detected by the force detector 13, is set as the individual force at newly added reference point.

Further, the reference point calculating units 10 and 10B automatically calculate the reference points, but, for example, the operator 6 may set the reference points in such a manner that a time point of passing through each of the meandering portion of the blood vessel 3, or a time point of passing through the branch portion may be set as the reference point, or the operator 6 may set the reference point.

Further, the above embodiments describe only the insertion direction, but the measurement can be conducted also for the rotating direction around the insertion direction with the similar method.

The above embodiments describe the catheter insertion as an example. In this description, when the insertion member is inserted into the vessel or pipe, the force at time the insertion member contacts with the vessel or pipe individually is calculated, and the embodiments produce the similar effect also in, for example, an endoscopic inspection for human bodies or in industrial endoscopes.

Though the present disclosure has been described above based on the above first to fifth embodiments and modification examples, the present disclosure should not be limited to the above-described first to fifth embodiments and modification examples. For example, the present disclosure also includes the following cases.

Part or entirety of each of the above-described force measurement apparatuses and control devices is actually a computer system that includes, for example, a microprocessor, ROM, RAM, hard disk unit, display unit, keyboard, mouse, and the like. A computer program is stored on the RAM or the hard disk unit. Functions of each of the apparatuses and devices can be achieved by the microprocessor operating according to the computer program. The computer program mentioned here is a combination of a plurality of instruction codes that indicate commands to a computer for achieving predetermined functions.

For example, each component can be implemented as a result that a program executing section (part/unit) such as a CPU reads and executes software programs recorded in a recording medium such as a hard disk or semiconductor memory. Here, software that implements a part or entirety of the apparatus and devices according to each of the above-mentioned embodiments and modification examples is a following program. That is to say, this program has a computer execute the sections (parts/units) defined in claims. The program has a computer execute the units/steps defined in claims. That is, such a program is a force measurement program that, when an insertion member that is a catheter or an endoscope is inserted into a living body vessel, measures a force at time the insertion member contacts with the living body vessel,

the program allowing a computer to function as:

an individual force calculation parameter determining unit that determines a time point a force generated during insertion of the insertion member into the living body vessel is individually measured or an insertion length at that time point as an individual force calculation parameter during the insertion of the insertion member into the living body vessel based on information about a force detected by a force detector that measures, from an outside of the living body vessel, the force generated during the insertion of the insertion member into the living body vessel; and

an individual force calculating unit that individually calculates the force generated during the insertion of the insertion member into living body vessel at each time point or each insertion length as an individual force based on information about the time point or the insertion length at that time point that is determined as the individual force calculation parameter by the individual force calculation parameter determining unit and the information about the force detected by the force detector.

In addition, it may be possible to execute the program by downloading it from a server or reading it from a predetermined storage medium (an optical disc such as a CD-ROM, a magnetic disc, a semiconductor memory, or the like).

Further, one or more computers can be used to execute the program. That is, centralized processing or distributed processing can be performed.

By properly combining the arbitrary embodiment(s) or modification example(s) of the aforementioned various embodiments and modification examples, the effects possessed by the embodiment(s) or modification example(s) can be produced.

### INDUSTRIAL APPLICABILITY

The above aspects of the present invention are useful as the force measurement apparatus and the measuring method, the master slave apparatus, the force measurement program, and the integrated electronic circuit, each of which measures a force generated at time of inserting an insertion member into a living body vessel.

The entire disclosure of Japanese Patent Application No.: 2012-154548 filed on Jul. 10, 2012, including specification, claims, drawings, and summary are incorporated herein by reference in its entirety.

Although the present disclosure has been fully described in connection with the embodiments thereof with reference to the accompanying drawings, it is to be noted that various changes and modifications are apparent to those skilled in the art. Such changes and modifications are to be understood as included within the scope of the present disclosure as defined by the appended claims unless they depart therefrom.

What is claimed is:

1. A force measurement apparatus that, when an insertion member that is a catheter or an endoscope is inserted into a living body vessel, measures a force at time when the insertion member contacts with the living body vessel, the apparatus comprising:
a force detector that measures, from an outside of the living body vessel, a force generated during the insertion of the insertion member into the living body vessel;
an individual force calculation parameter determining unit that determines a time point when the force generated during the insertion of the insertion member into the living body vessel is individually measured or an insertion length at that time point as an individual force calculation parameter based on information about the force detected by the force detector during the insertion of the insertion member into the living body vessel; and
an individual force calculating unit that individually calculates the force generated during the insertion of the insertion member into living body vessel at each time point or each insertion length as an individual force based on the time point or information about the insertion length at that time point and the information about the force detected by the force detector as the individual force calculation parameter determined by the individual force calculation parameter determining unit.

2. The force measurement apparatus according to claim 1, wherein when the insertion member is inserted into the living body vessel, the individual force calculation parameter determining unit determines a time point or an insertion length where displacement of the force is a predetermined threshold value or more, as the individual force calculation parameter at each predetermined insertion length,
the individual force calculating unit adds a value, which is obtained by dividing a value, which is obtained by subtracting information about the force at the immediately preceding time point or at an insertion length at that time point from the information about the force detected by the force detector at a measurement time point or at an insertion length at that time point, by a number of the time points or the insertion lengths determined until the measurement time point or the insertion length, to the individual force at each time point or each insertion length.

3. The force measurement apparatus according to claim 1, further comprising: a correcting unit that, when after the insertion member is inserted into the living body vessel and is once partially pulled back, the insertion member is again inserted into the living body vessel, makes a correction so that the time points or the insertion lengths, which are already determined by the individual force calculation parameter determining unit between a pulling-back start time point or an insertion length at that time point and a reinsertion time point or an insertion length at that time point, are deleted, wherein
the individual force calculating unit calculates individual forces based on the time point or the insertion length corrected by the correcting unit.

4. The force measurement apparatus according to claim 2, further comprising: a correcting unit that, when after the insertion member is inserted into the living body vessel and is once partially pulled back, the insertion member is again inserted into the living body vessel, makes a correction so that the time points or the insertion lengths, which are already determined by the individual force calculation parameter determining unit between a pulling-back start time point or an insertion length at that time point and a reinsertion time point or an insertion length at that time point, are deleted, wherein
the individual force calculating unit calculates individual forces based on the time point or the insertion length corrected by the correcting unit.

5. The force measurement apparatus according to claim 1, further comprising a force deciding unit that, when information about forces of the predetermined threshold value or more in the information about the individual forces calculated by the individual force calculating unit is present, decides that a load is generated in the living body vessel or the insertion member.

6. The force measurement apparatus according to claim 2, further comprising a force deciding unit that, when information about forces of the predetermined threshold value or more in the information about the individual forces calculated by the individual force calculating unit is present, decides that a load is generated in the living body vessel or the insertion member.

7. The force measurement apparatus according to claim 1, further comprising: an imaging device that images an image of a portion of the living body vessel into which the insertion member is inserted; and
a decided result notification unit that adds the individual forces calculated by the individual force calculating unit or a decided result decided by the force deciding unit to the image obtained by imaging the living body vessel or the insertion member so as to display the image.

8. The force measurement apparatus according to claim 2, further comprising: an imaging device that images an image of a portion of the living body vessel into which the insertion member is inserted; and
a decided result notification unit that adds the individual forces calculated by the individual force calculating unit or a decided result decided by the force deciding unit to the image obtained by imaging the living body vessel or the insertion member so as to display the image.

9. The force measurement apparatus according to claim 1, further comprising: an output unit that notifies an operator of the individual forces calculated by the individual force calculating unit or the decided result decided by the force deciding unit as a sound or an image.

10. The force measurement apparatus according to claim 2, further comprising: an output unit that notifies an operator of the individual forces calculated by the individual force calculating unit or the decided result decided by the force deciding unit as a sound or an image.

11. The force measurement apparatus according to claim 1, further comprising:
a notification information determining unit that determines information to be notified based on the decided result decided by the force deciding unit;
an imaging device that images an image of the portion of the living body vessel into which the insertion member is inserted based on the notification information determined by the notification information determining unit;
an imaging device controller that controls the imaging device; and
a decided result notification unit that adds the notification information determined by the notification information determining unit to the image imaged by the imaging device under control of the imaging device controller so as to display the image.

12. The force measurement apparatus according to claim 2, further comprising:
a notification information determining unit that determines information to be notified based on the decided result decided by the force deciding unit;
an imaging device that images an image of the portion of the living body vessel into which the insertion member is inserted based on the notification information determined by the notification information determining unit;
an imaging device controller that controls the imaging device; and
a decided result notification unit that adds the notification information determined by the notification information determining unit to the image imaged by the imaging device under control of the imaging device controller so as to display the image.

13. A force measurement method for, when an insertion member that is a catheter or an endoscope is inserted into a living body vessel, measuring a force at time the insertion member contacts with the living body vessel, the method comprising;
measuring, from an outside of the living body vessel, a force generated during insertion of the insertion member into the living body vessel, using a force detector;
determining a time point when the force generated during the insertion of the insertion member into the living body vessel is individually measured or an insertion length at that time point as an individual force calculation parameter based on information about the force detected by the force detector during the insertion of the insertion member into the living body vessel, using an individual force calculation parameter determining unit; and
individually calculating the force generated during the insertion of the insertion member into the living body vessel at each time point or at each insertion length as an individual force based on information about the time point or the insertion length at that time point determined as the individual force calculation parameter by the individual force calculation parameter determining unit and the information about the force detected by the force detector, using an individual force calculating unit.

14. A computer-readable recording medium including a force measurement program that, when an insertion member that is a catheter or an endoscope is inserted into a living body vessel, measures a force at time the insertion member contacts with the living body vessel,
the program allowing a computer to function as:
an individual force calculation parameter determining unit that determines a time point a force generated during insertion of the insertion member into the living body vessel is individually measured or an insertion length at that time point as an individual force calculation parameter during the insertion of the insertion member into the living body vessel based on information about a force detected by a force detector that measures, from an outside of the living body vessel, the force generated during the insertion of the insertion member into the living body vessel; and
an individual force calculating unit that individually calculates the force generated during the insertion of the insertion member into living body vessel at each time point or each insertion length as an individual force based on information about the time point or the insertion length at that time point that is determined as the individual force calculation parameter by the individual force calculation parameter determining unit and the information about the force detected by the force detector.
